(19) United States
(12) Patent Application Publication
(10) Pub. No.: US 2008/0161643 A1
(43) Pub. Date: Jul. 3, 2008

(54) CAPSULE MEDICAL APPARATUS AND CONTROL METHOD FOR CAPSULE MEDICAL APPARATUS

(75) Inventors: Akio Uchiyama, Yokohama-shi (JP); Takeshi Yokoi, Tokyo (JP); Wataru Ono, Toyko (JP); Jun Hasegawa, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/042,948

(22) Filed: Mar. 5, 2008

Related U.S. Application Data
(63) Continuation-in-part of application No. 10/438,484, filed on May 15, 2003.

(30) Foreign Application Priority Data
May 16, 2002 (JP) 2002142099

(51) Int. Cl. A61B 1/04

(52) U.S. Cl. 600/109

(57) ABSTRACT

A capsule medical apparatus has a space setting unit which designates a space in vivo and a capsule which is inserted or swallowed in vivo. Further, the capsule medical apparatus has a recognizing unit which recognizes whether or not the capsule exists in the space set by the space setting unit and a control unit which controls a state of the capsule based on an output from the recognizing unit. Thus, the capsule reaches the space, then, the state of the capsule is controlled, and medical activity is performed.

### CROSS REFERENCE TO RELATED APPLICATION

This application is a continuation-in-part of U.S. application Ser. No. 10/438,484 filed on May 15, 2003 which claims benefit of Japanese Application No. 2002-142099 filed on May 16, 2002, the entire contents of which are incorporated herein by this reference.

### BACKGROUND OF THE INVENTION

1. Field of the Invention

The present invention relates to a capsule medical apparatus which is inserted or swallowed in the coelom for a medical activity or the acquisition of vital information.

2. Description of the Related Art

Japanese Unexamined Patent Application Publication No. 2001-179700 discloses a position detecting apparatus.

In the above-mentioned related art, a micromachine and a system for controlling the movement of the micromachine are disclosed. The micromachine comprises: a magnetic field generating unit for generating a rotating magnetic field; a robot main body for receiving the rotating magnetic field and obtaining propelling power by rotation; a position detecting unit for detecting the position of the robot main body, and magnetic field changing means for changing the direction of the rotating magnetic field generated by the magnetic field generating unit so that the robot main body reaches a target destination.

### SUMMARY OF THE INVENTION

A capsule medical apparatus comprises: a capsule which is inserted or swallowed in vivo and a space setting unit which designates a space in vivo previously before the capsule is inserted or swallowed in vivo. Further, the capsule medical apparatus comprises: a recognizing unit which recognizes whether or not the capsule exists in the space set by the space setting unit; and a control unit which controls a state of the capsule based on an output from the recognizing unit.

Further, a capsule medical apparatus comprising a capsule having a vital information detecting unit for obtaining vital information, inserted in vivo, and an extracorporeal unit arranged in vitro, the capsule medical apparatus comprises: a position detecting unit which detects a position of the capsule; a space setting unit which designates a space in vivo previously before the capsule is inserted or swallowed in vivo; a comparing unit which compares information on the capsule position from the position detecting unit with the space set by the space setting unit and outputs a signal corresponding to a comparison result; and a control unit which controls a state of the capsule based on a signal output from the comparing unit.

### BRIEF DESCRIPTION OF THE DRAWINGS

FIG. 1A is a block diagram showing the entire structure of a capsule medical apparatus according to a first embodiment of the present invention;

FIG. 1B is a diagram showing the structure of a capsule;

FIG. 2 is a flowchart for explaining the operation of the capsule medical apparatus;

FIG. 3A is a diagram showing a file format for storing image information and positional information by an extracorporeal unit;

FIG. 3B is a diagram showing a file format for separately storing the image information and the positional information according to a modification of the first embodiment;

FIG. 4 is a diagram showing a display example of a picked-up image on a monitor;

FIG. 5 is a diagram showing a display example of a picked-up image and the position thereof by using a ratio according to a second embodiment of the present invention;

FIG. 6 is a block diagram showing the entire structure of a capsule medical apparatus according to a third embodiment of the present invention;

FIGS. 7A and 7B are diagrams showing formats of image information and information on signal intensity stored in an extracorporeal unit;

FIG. 8 is a diagram showing a capsule having a magnet according to a fourth embodiment of the present invention;

FIG. 9 is a block diagram showing a capsule medical apparatus according to a fifth embodiment of the present invention;

FIGS. 10A and 10B are diagrams showing a side surface and a front surface of the capsule;

FIG. 11A is a schematic diagram showing a rotating magnetic field generating device;

FIG. 11B is an explanatory diagram schematically showing the operation of a rotating magnetic field generated by the rotating magnetic field generating device;

FIG. 12 is a block diagram showing the entire structure of a capsule medical apparatus according to a sixth embodiment of the present invention;

FIG. 13 is a block diagram showing the entire structure of a capsule medical apparatus according to a seventh embodiment of the present invention;

FIG. 14 is a block diagram showing the entire structure of a capsule medical apparatus according to an eighth embodiment of the present invention;

FIG. 15 is a block diagram showing the entire structure of a capsule medical apparatus according to a ninth embodiment of the present invention;

FIG. 16 is a schematic diagram showing a capsule according to a tenth embodiment of the present invention;

FIG. 17 is a schematic diagram showing a capsule according to a first modification of the tenth embodiment;

FIG. 18 is a schematic diagram showing a capsule according to a second modification of the tenth embodiment;

FIG. 19 is a schematic diagram showing a capsule according to a third modification of the tenth embodiment;

FIG. 20 is a schematic diagram showing a capsule according to an eleventh embodiment of the present invention;

FIG. 21 is a diagram showing the directivity of electric wave through a transmitting antenna arranged to the body surface of a patient according to the eleventh embodiment; and

FIG. 22 is a schematic diagram showing a capsule according to a modification of the eleventh embodiment.

### DESCRIPTION OF THE PREFERRED EMBODIMENTS

Hereinbelow, embodiments of the present invention will be described with reference to the drawings.

### First Embodiment

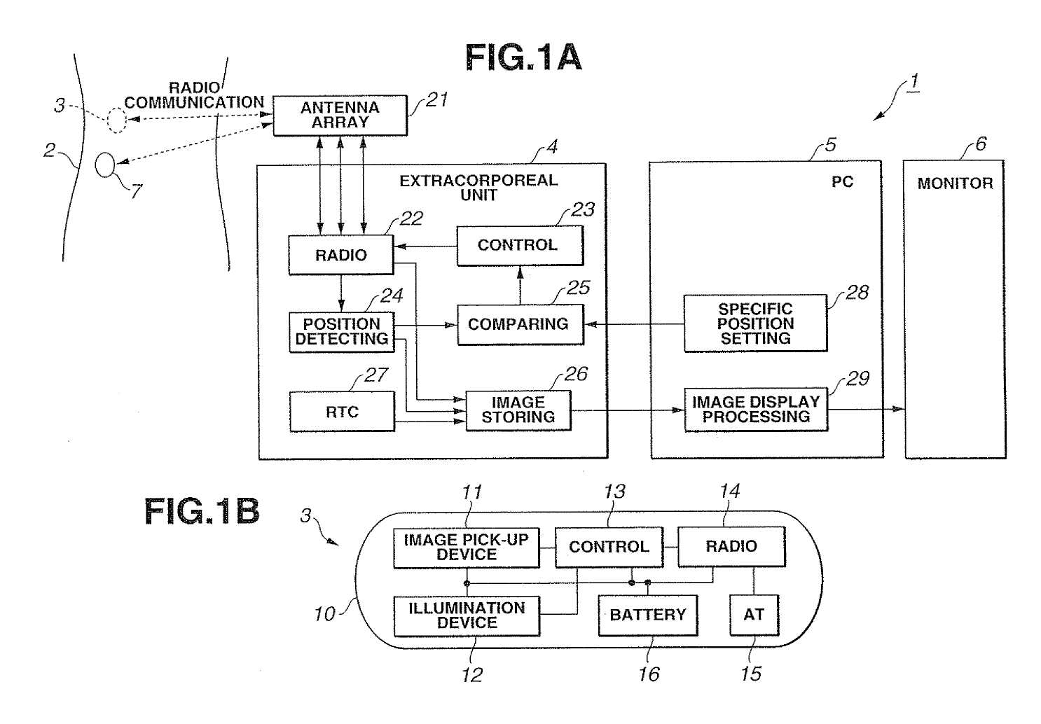

A first embodiment of the present invention will be described with reference to FIGS. 1A to 4.

Referring to FIG. 1A, a capsule medical apparatus 1 according to the first embodiment comprises: a capsule 3 which obtains vital information in the body of a patient 2 (e.g., optically captured image information according to the first embodiment); an extracorporeal unit 4 which is arranged in vitro, obtains the vital information through communication with the capsule 3, and detects the spatial position of the capsule 3; a personal computer (abbreviated to a PC in FIG. 1A) 5 which is detachably connected to the extracorporeal unit 4 and sets the operation for capturing the vital information stored by the extracorporeal unit 4 and for obtaining the vital information; a monitor 6 which is connected to the PC 5 and displays the vital information, etc.; and a reference marker 7 which is attached to a reference position on an arbitrary position of the body surface of the patient 2 and outputs a transmitting signal at the reference position to improve the accuracy for detecting the position.

Referring to FIG. 1B, the capsule 3 inserted in the body of the patient 2 by the deglutition from the mouth comprises: an image pick-up device 11 which picks up an image in a capsule container 10; an illumination device 12 which illuminates light for the image pick-up operation of the image pick-up device 11; a control circuit 13 which controls the image pick-up device 11, the illumination device 12, etc. and performs signal processing of a signal captured by the image pick-up device 11; a radio circuit 14 which transmits the vital information (specifically, image information) captured by the image pick-up device 11 via the control circuit 13; an antenna (abbreviated to an AT in FIG. 1B) 15 which is connected to the radio circuit 14 and transmits the image information by radio (electric wave) to the extracorporeal unit 4; and a battery 16 which supplies power to operate the image pick-up device 11, the illumination device 12, the control circuit 13, and the radio circuit 14.

The radio circuit 14 transmits a signal for detecting the position from the antenna 15 until an instruction for starting the image pick-up operation (photographing). In this case, the radio circuit 14 transmits the signal with a predetermined amplitude and, on the extracorporeal unit 4 side, a plurality of antennas arranged to the different positions near the body surface are switched and thereby the transmitted signal is received so as to detect the spatial position of the capsule 3 based on the intensity of the receiving signal.

The antenna 15 receives the signal transmitted by radio from the extracorporeal unit 4, demodulates the receiving signal by the radio circuit 14, and transmits the demodulated signal to the control circuit 13. When the control circuit 13 determines that the receiving signal indicates a command for starting to capture the image information (starting the image pick-up operation), it drives the image pick-up device 11 and the illumination device 12 to start the image pick-up operation. When the control circuit 13 determines that the receiving signal indicates a command for ending the image pick-up operation, the image pick-up operation ends.

The control circuit 13 includes a memory such as a ROM (not shown), for storing information corresponding to codes of the commands for starting and ending the image pick-up operation. When the signal is received from the extracorporeal unit 4, the control circuit 13 determines whether or not the receiving signal is the command and controls the circuits in the capsule 3 to execute the operation in accordance with the determination result.

On the other hand, the extracorporeal unit 4 comprises: an antenna array 21 for radio communication with the capsule 3; a radio circuit 22 which is connected to a plurality of antennas forming the antenna array 21 and modulates and demodulates the signal for radio communication; a control circuit 23 which is connected to the radio circuit 22 and controls the operation; a position detecting circuit 24 which is connected to the radio circuit 22 and detects the position of the capsule 3; a comparing circuit 25 which compares positional information from the position detecting circuit 24 with setting information of a position set by the PC 5 previously before the capsule 3 is inserted in vivo; an image storing device 26 which stores image information received by the radio circuit 22; and a real-time clock (RTC) 27 which outputs date information for storing the image information in the image storing device 26.

The image storing device 26 stores not only the image information received by the radio circuit 22 but also the positional information detected by the position detecting circuit 24.

The PC 5 comprises: a position setting unit 28 which sets a spatial area that is set previously before the capsule 3 is inserted in vivo, for starting the image pick-up operation by the capsule 3 (also referred to as a spatial position because this area can be set to be small depending on an error for detecting the position and can be assumed as the position when the detecting accuracy of the position detecting circuit 24 is high) and which further sets a spatial area (position) for ending the image pick-up operation; and an image display processing unit 29 which captures the image information and the positional information from the image storing device 26 and displays the image information and the positional information.

In case the small intestine in the patient 2 is examined by using the capsule 3 by the position setting unit 28, the duodenum is designated as a first spatial position for starting the image pick-up operation by the position setting unit 28 and the appendix is designated as a second spatial position for ending the image pick-up operation, previously before the capsule 3 is inserted in vivo.

The positional data on the two setting positions is transferred to the comparing circuit 25 in the extracorporeal unit 4, and is stored as information on the reference position into the memory (not shown).

The image display processing unit 29 captures the image information and the positional information stored by the image storing device 26. Referring to FIG. 4, the image display processing unit 29 displays both the image picked-up by the capsule 3 and the positional image of the positional information detected by the position detecting circuit 24.

When the personal computer 5 sets the spatial position, a patient data input unit is provided to input data of the patient 2 for the examination using the capsule 3. The patient data is stored in the image storing device 26 of the extracorporeal unit 4 before storing the image information. The image storing device 26 stores a plurality of pieces of image information which are picked up by the capsule 3, after the patient data.

In other words, the image storing device 26 in the extracorporeal unit 4 stores the image information in association with the patient information.

In the case of setting the spatial position by the position setting unit 28, the positions for starting and ending the image pick-up operation are set with reference to the information on the reference position of the reference position marker 7, examination information from an ultrasonic diagnostic device or an X-ray device, and static data on the organ position depending on the body shape.

As a result of the above-mentioned setting of the spatial position (area) previously before the capsule 3 is inserted in vivo, the position is accurately set and the position is precisely detected by the signal for detecting the position from the capsule 3.

According to the first embodiment, the spatial positions for starting and ending the image pick-up operation are set and the control operation is performed so that the vital information (specifically, the image information) is automatically obtained only between the positions. Thus, unnecessary power consumption is suppressed in the battery 16 incorporated in the capsule 3 and the vital information is obtained at the position for the desired vital information.

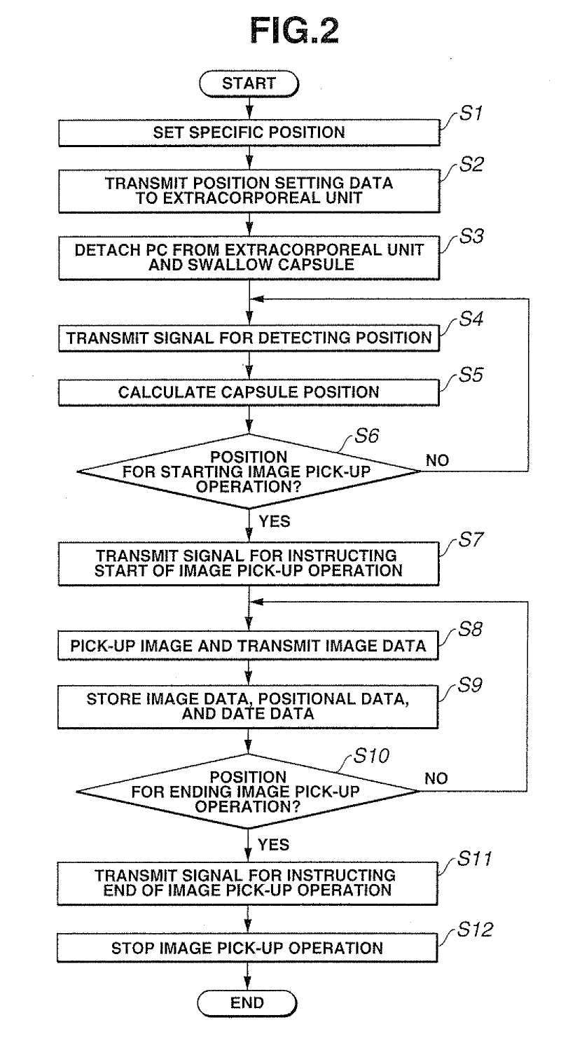

The operation with the above-described structure will be described according to the first embodiment with a flowchart shown in FIG. 2.

In step S1, the positions are set by the PC 5. Namely, the position setting unit 28 sets the positions for starting and ending the image pick-up operation and transmits the positional information to the extracorporeal unit 4. Concretely, in the case of examining the small intestine, the position of the duodenum is set as the position for starting the image pick-up operation and the position of the appendix is set as the position for ending the image pick-up operation.

In the case of designating the position for starting the image pick-up operation, a plurality of positions for designating an area near the duodenum may be designated as the position for starting the image pick-up operation in consideration of the position detecting error and the comparing circuit 25 may compare and determine whether or not the position is the position for starting the image pick-up operation depending on whether or not the position is included in the plurality of positions.

After ending the setting of the positions for starting and ending the image pick-up operation, in step S2, the position setting data is transmitted to the extracorporeal unit 4, and the extracorporeal unit 4 stores the setting data in the memory in the comparing circuit 25. In step S3, the PC 5 is detached from the extracorporeal unit 4 and the patient 2 swallows the turned-on capsule 3.

Then, in step S4, the capsule 3 transmits the signal for detecting the position. The extracorporeal unit 4 switches the plurality of antennas forming the antenna array 21, demodulates the signal by the radio circuit 22, and transmits the demodulated signal to the position detecting circuit 24.

In step S5, the position detecting circuit 24 calculates the position of the capsule 3, and transmits the calculated positional data to the comparing circuit 25.

In step S6, the comparing circuit 25 determines whether or not the calculated positional data matches (overlaps to) the positional data at the position for starting the image pick-up operation of the position setting data, within threshold value. When NO in step S6, the processing routine returns to step S4 whereupon the position is calculated based on the signal for detecting the position transmitted from the capsule 3 and the processing for determining whether or not the positional data matches the positional data at the position for starting the image pick-up operation is repeated.

When the capsule 3 reaches the position for starting the image pick-up operation, the calculated positional data matches the setting data as the position for starting the image pick-up operation stored in the memory in the comparing circuit 25 within the threshold value. In this case, in step S7, the comparing circuit 25 in the extracorporeal unit 4 transmits the matching result to the control circuit 23, and the control circuit 23 transmits a signal for instructing the start of the image pick-up operation to the capsule 3 via the radio circuit 22.

On the capsule 3 side, the control circuit 13 reads the contents of the command of the signal for instructing the start of the image pick-up operation by comparing it with the command cords previously stored in the storing unit. Then, in step S8, both of the illumination device 12 and the image pick-up device 11 are driven, and the control circuit 13 starts the image pick-up operation and transmits the image pick-up data and the positional data. In this case, the illumination device 12 and the image pick-up device 11 are driven for a predetermined period.

That is, the energy consumption of the battery 16 is saved because the image pick-up operation is not necessary until the capsule 3 reaches the position for starting the image pick-up operation.

On the extracorporeal unit 4 side, the image data received via the radio circuit 22 is inputted to the image storing device 26. In step S9, the image storing device 26 stores the image data, the positional data detected by the position detecting circuit 24, and date data from the RTC 27.

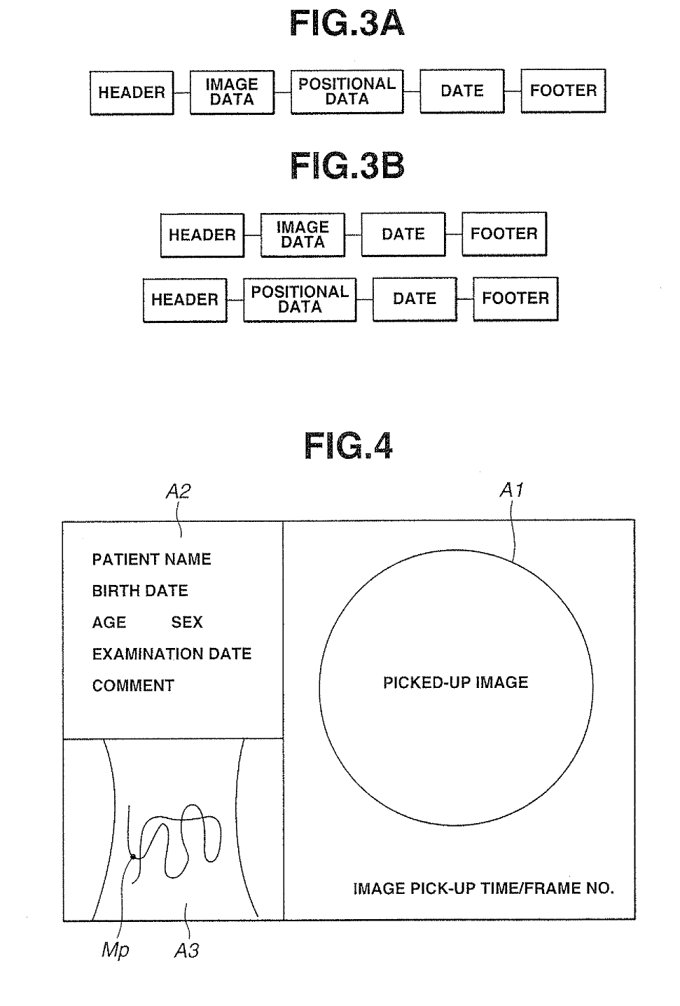

Referring to FIG. 3A, the image storing device 26 stores the image information in order of a header, the image data, the positional data, and a footer. As mentioned above, the capsule 3 reaches the position for starting the image pick-up operation and, then, the capsule 3 starts the image pick-up operation and transmits the image pick-up data and the position signal to the extracorporeal unit 4. The extracorporeal unit 4 stores the image data, the position data, and the date data in association therewith.

The positional data detected by the position detecting circuit 24 is transmitted to the comparing circuit 25. In step S10, the comparing circuit 25 determines whether or not the positional data transmitted from the position detecting circuit 24 matches the positional data at the position for ending the image pick-up operation within the threshold value. When NO in step S10, the processing routine returns to step S8 whereupon the image pick-up operation continues.

When the capsule 3 reaches the position for ending the image pick-up operation, the positional data is detected because it matches the setting data stored in the memory in the comparing circuit 25. A detection result is transmitted to the control circuit 23.

In step S11, the control circuit 23 transmits the instructing signal for ending the image pick-up operation to the capsule 3. In step S12, the capsule 3 receives the instructing signal and stops the image pick-up operation.

After that, the extracorporeal unit 4 is removed from the patient 2 and is connected to the PC 5. The image information containing the image data stored in the image storing device 26 of the extracorporeal unit 4 is captured in the image display processing unit 29, and the captured information is displayed on the monitor 6.

Referring to FIG. 4, the monitor 6 displays the picked-up image of the capsule 3 on an image display area A1 on the right side of the display surface, the patient data on a patient data display area A2 on the upper left side, and the shape of the main portion in the patient 2 and the position of the capsule 3 calculated by the position detecting circuit 24 in a positional data display area A3 on the down left side.

The picked-up image is displayed together with an image pick-up time and a picked-up frame No. on the bottom.

As shown in FIG. 4, the position of the capsule 3 is displayed as a locus which linearly connects the position of the capsule 3 on time-series on the positional data display area A3 on the down left side, thereby easily grasping the shape of the organ.

Further, the picked-up image and a positional marker Mp indicating the position of the capsule 3 may be displayed on the positional data display area A3 shown in FIG. 4 from the information shown in FIG. 3A. In addition, the positional data is collected and the position locus is displayed as shown in FIG. 4. Then, the position corresponding to the picked-up image displayed on the locus is displayed by the positional marker Mp.

Although the image pick-up operation ends in step S12 in FIG. 2, the position is continuously detected because the image pick-up operation ends while power remains in the capsule 3, Consequently, easily, the time for removing the capsule 3 is confirmed and predicted.

With the operation according to the first embodiment, the extracorporeal unit 4 first needs to be connected to the PC 5 and, however, the extracorporeal unit 4 can be detached from the PC 5 after designating the picked-up area and the patient 2 freely acts.

Further, according to the first embodiment, the image pick-up operation is not necessary until the capsule 3 reaches the observed portion and the image pick-up operation starts after confirming the reach to the observed portion. In other words, because the capsule 3 automatically performs a medical activity only in spaces designated previously before the capsule 3 is inserted or swallowed in vivo. Thus, the image is efficiently picked up by the capsule 3 and the driving for a long time is possible at the target position.

The picked-up image is displayed on the monitor 6 via the PC 5 after the image pick-up operation and the operator confirms the picked-up image. Since the image at a non-observed position does not need to be confirmed, the diagnosis becomes efficient.

The image is observed while grasping the organ position observed by the capsule, by displaying both of the capsule position and the image.

The image from the capsule 3 is stored while the time information is obtained from the RTC 27 and is added to the image data. Therefore, the image pick-up time is certainly stored.

Although the image data is stored in the image storing device 26 by the format shown in FIG. 3A as mentioned above, it may be stored by a format shown in FIG. 3B.

Namely, since the image acquisition and the position detection are at different timings, two pieces of information may be stored as information having individual correlation therewith.

Referring to FIG. 3B, the image information sequentially has a header, image data, date, and footer in order thereof. The positional information sequentially has the header, positional data, date, and footer in order thereof. Accordingly, the image information and the positional information have different correlations with the image pick-up time. The positional information in this case may be obtained by adding the latest data upon position detection.

### Second Embodiment

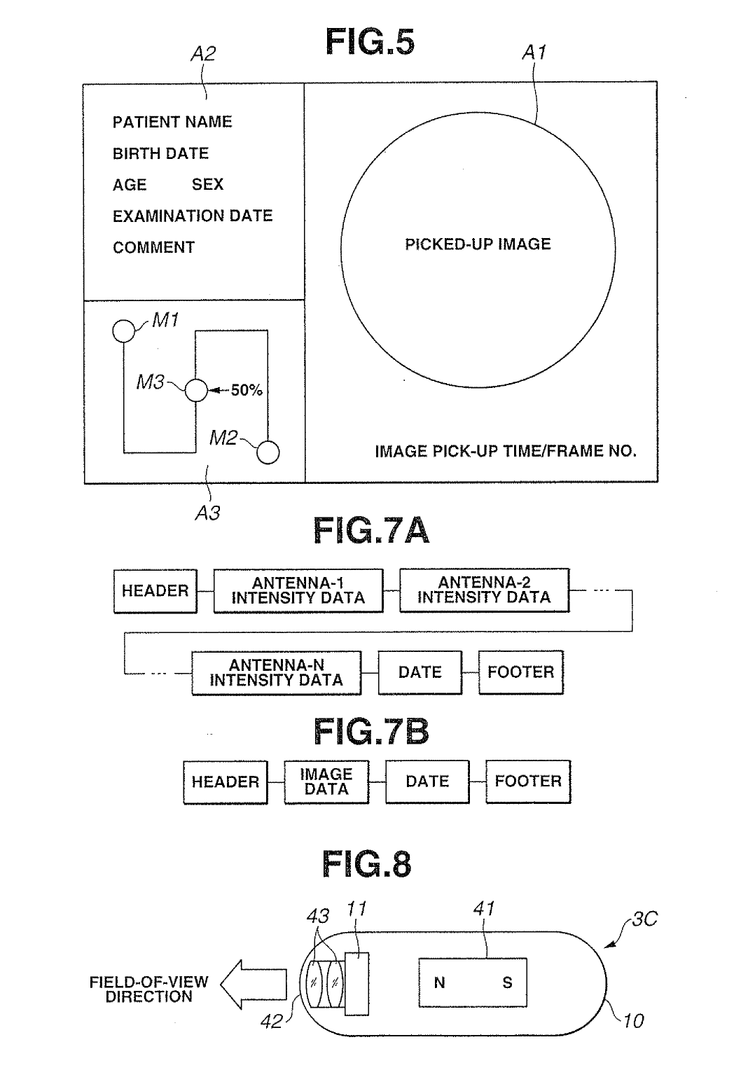

Next, a second embodiment of the present invention will be described with reference to FIG. 5. The second embodiment is similar to the first embodiment. However, a partially different structure is the image display processing of the image display processing unit for the image display processing on the PC 5 side.

According to the second embodiment, the PC 5 captures the image information from the image storing device 26 in the extracorporeal unit 4 so as to easily display which ratio (e.g., by percent) of the picked-up image displayed in the image display area A1 to the entire length of an observing range including a first marker M1 indicating the position for starting the image pick-up operation and a second marker M2 indicating the position for ending the image pick-up operation, as shown in FIG. 5.

That is, not only the picked-up image is displayed on the monitor 6 but also it is displayed which ratio of the position for obtaining the picked-up image to entire length of the observing range. When the picked-up image is found as the target image which captures the disease portion, a doctor for the diagnosis thereof easily grasps the position of the disease position by displaying the ratio (e.g., 50% in FIG. 5) of the position for capturing the picked-up image at the disease portion (e.g., M3 in FIG. 5) to the entire length.

Additional information such as an identification No. is added to the picked-up target image as the disease portion by using the keyboard of the PC 5 so as to easily search for the information of the picked-up image.

The picked-up image is confirmed by inputting an instruction for displaying the picked-up image with the additional information. Thus, the picked-up image is searched and displayed, and the position for capturing the picked-up image is displayed with the ratio to the entire length.

According to the second embodiment, not only the picked-up image is displayed but also the ratio of the position for capturing the picked-up image to the entire length of the observing range is displayed. Accordingly, it is possible to provide a capsule medical apparatus which can easily grasp the position for catching the disease portion and can promptly perform the diagnosis.

Since the position detecting unit is provided according to the second embodiment, the ratio of the position of the picked-up image displayed on the monitor 6 to the entire of the observing range is displayed. However, the display operation may be used according to the following modification of the second embodiment.

In other words, according to the modification, the positional information from the position detecting unit is not used. The entire time for the passage through the entire observing range is simply calculated based on time information on the position for starting the image pick-up operation and time information on the position for ending the image pick-up operation. Accordingly, it may be displayed which ratio of the position for capturing the picked-up image displayed on the monitor 6 to the entire observing range based on the calculated time information.

In this case, the positional marker Mp (displayed in the image display area A1) in the positional data display area A3 in FIG. 5 displays which ratio (percent) of the position on time base to the time difference between the passage time of the first marker position M1 and the passage time of the second marker position M2, the position on the time base of the picked-up image.

In other words, a time bar is displayed while the first marker position M1 is the start point and the second marker position M2 is the end point. Further, the image pick-up time of the picked-up image displayed in the image display area A1 may be displayed on the time bar. This display operation format can widely be applied to the case in which the position detecting unit is not provided and the date information from the RTC 27 is supplied (according to a ninth embodiment of the present invention, which will be described later).

### Third Embodiment

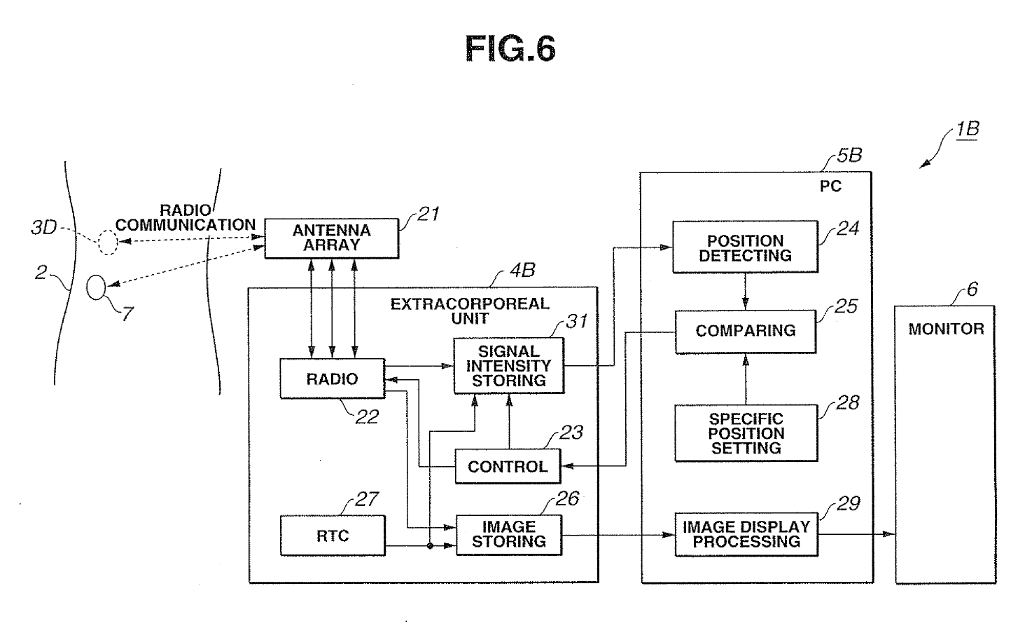

Next, a third embodiment of the present invention will be described with reference to FIGS. 6 to 7B.

Referring to FIG. 6, a capsule medical apparatus 1B according to the third embodiment of the present invention is obtained by partially changing the structure of the capsule medical apparatus 1 shown in FIG. 1A according to the first embodiment.

Specifically, the position detecting circuit 24 is provided in the extracorporeal unit 4 to detect (calculate) the position. However, the position detecting function shifts to the PC 5B side.

That is, in place of the extracorporeal unit 4 in the capsule medical apparatus 1 and the PC 5 according to the first embodiment, the capsule medical apparatus 1B uses an extracorporeal unit 4B and a personal computer (PC) 5B according to the third embodiment. Further, the extracorporeal unit 4B is connected to the radio circuit 22, and incorporates a signal intensity storing circuit 31 having a function for storing information on the signal intensity upon switching an antenna of the antenna array 21 and receiving the signal. Furthermore, the PC 5B incorporates the position detecting circuit 24 arranged to the extracorporeal unit 4 and the comparing circuit 25.

The extracorporeal unit 4B stores in the signal intensity storing circuit 31, the information on the signal intensity upon switching the plurality of antennas forming the antenna array 21 and receiving the signals, by using the radio circuit 22. The signal intensity storing circuit 31 may output the information on the signal intensity to the position detecting circuit 24 of the PC 5B without storing the information until the capsule 3 reaches the position for starting the image pick-up operation.

Specifically, when the extracorporeal unit 4B is connected to the PC 5B, the signal intensity storing circuit 31 outputs to the position detecting circuit 24 of the PC 5B, the received signals through the switching of the plurality of antennas, namely, signals having N pieces of intensity data of an antenna I (=1 to N assuming that the number of antennas of the antenna array 21 is N).

The position detecting circuit 24 calculates the position of the capsule 3 based on the N pieces of intensity data, and outputs the calculated position to the comparing circuit 25. The comparing circuit 25 determines whether or not the calculated position matches the positional data of the position for starting the image pick-up operation set by the position setting unit 28 within the threshold value, and outputs the comparison result to the control circuit 23 in the extracorporeal unit 4B.

When the comparison result indicates that the calculated position matches the position for starting the image pick-up operation within the threshold value, the control circuit 23 transmits the signal for instructing the start of the image pick-up operation via the radio circuit 22 and the capsule 3 starts the image pick-up operation, similarly to the first embodiment.

After that, the extracorporeal unit 4B can be detached from the PC 5B. When the image pick-up operation starts, the control circuit 23 transmits the control signal to the signal intensity storing circuit 31, and further stores the signal data on the signal intensity and the date data from the RTC 27 in a format, such as information on the signal intensity shown in FIG. 7A.

That is, the header, the intensity data on the antenna 1, the intensity data on the antenna 2, . . . , the intensity data on the antenna N, the date data, and the footer are sequentially stored as the information on the signal intensity in order thereof.

The capsule 3 performs the image pick-up operation and transmits the picked-up image. The extracorporeal unit 4B receives the image data. The image storing device 26 stores the image data in a format of the image information shown in FIG. 7B. This format is similar to that of the image information shown in FIG. 3B.

After the examination, the data is read to the PC 5B from the extracorporeal unit 4B, then, the positional information is obtained from the information on the signal intensity by using the position detecting circuit 24, and the obtained positional information is displayed in correlation to the image information.

For example, the positional information can be displayed as shown in FIG. 4 or 5.

The communication between the capsule 3 and the extracorporeal unit 4B is performed by using the electric waves which passes through the anatomy. Since the anatomy highly absorbs the electro-magnetic waves, a plurality of antennas forming the antenna array 21 are arranged near the patient 2 so as to preferably ensure the communicating state between the capsule 3 and the extracorporeal unit 4B. The antennas are substantially touched to the anatomy, thereby arranging the antennas without producing the boundary of the electromagnetic characteristics between the capsule 3 and the extracorporeal unit 4B. As a result, the positional information is simply calculated based on the data on signal intensity. Further, the precision for measuring the position is improved.

Since the attenuation of the electro-magnetic waves is large in the anatomy, all the signals do not sufficiently have the receiving sensitivity.

The data with strong signal intensity is preferentially used for the detection and calculation of the position. The data with weak signal intensity approximate to the noise level is not used for the detection of the position. Accordingly, the precision for detecting the position is improved.

With the above-mentioned structure and operations according to the third embodiment, the PC 5B performs the processing with a relatively large load and large power consumption for calculating the position, which is executed in the extracorporeal unit 4B. Thus, the structure of the extracorporeal unit 4B is simplified and costs are reduced. Further, the weight is reduced, the power consumption is low, and the using time is extended.

### Fourth Embodiment

Next, a fourth embodiment of the present invention will be described with reference to FIG. 8. According to the fourth embodiment, the structure is obtained by further providing two three-dimensional magnetic sensor units disclosed in Japanese Unexamined Patent Application Publication No. 2001-179700 or the following position detecting unit using two three-axial coils, and by furthermore providing a magnet as a capsule 3C so that two poles of the magnet 41 are in parallel with a field-of-view direction of the capsule 3C as shown in FIG. 8.

For example, an objective lens (image pick-up lens) 43 is arranged so that it faces the inside of an observation window 42 formed at a semispherical-shaped end portion of the capsule container 10. The magnet 41 having N and S poles is arranged in the direction along the field-of-view direction of the image pick-up device 11 arranged at the image-forming position. The capsule 3C includes not only the magnet 41 but also the illumination device 12 shown in FIG. 1B.

The capsule 3C is structured as mentioned above, thereby confirming the position and observing direction of the capsule 3C.

The detection of the position and direction of the capsule 3C needs a position detecting unit which detects six degrees of freedom. This is realized by using two sets of the three-axial coils.

Further, the two sets of three-axial coils are arranged to be touched to the body surface of the patient. Consequently, the attenuation characteristics of electro-magnetic waves become constant between the capsule 3C and the three-axial coils, and the precision for detecting the position is improved. Additionally, the same advantages as those according to the second or third embodiment are obtained.

### Fifth Embodiment

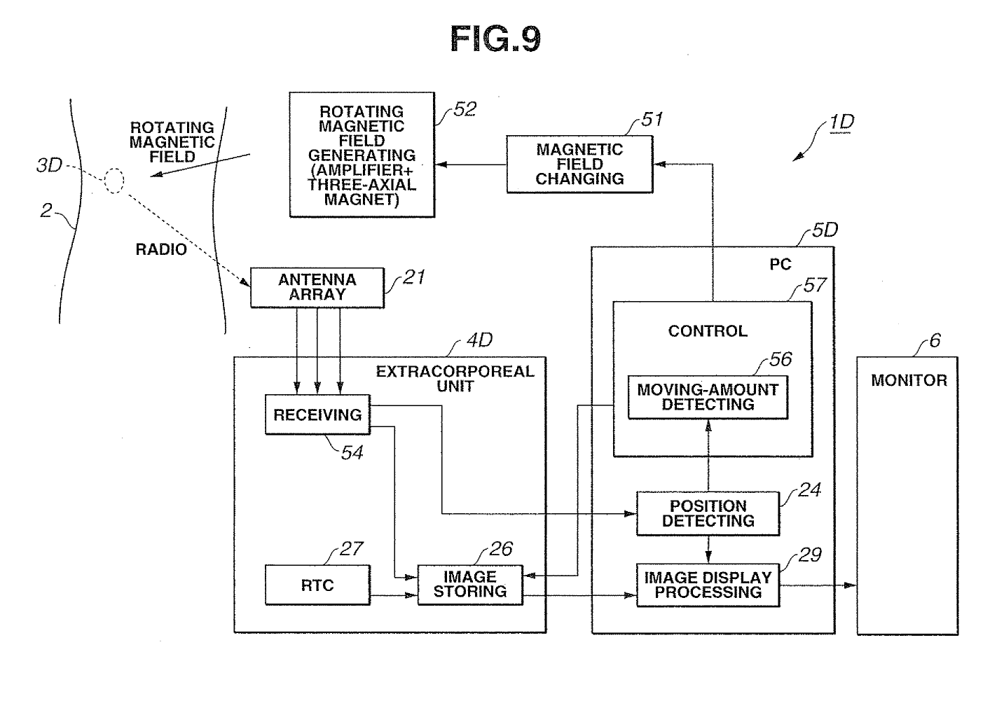

A fifth embodiment of the present invention will be described with reference to FIGS. 9 to 11B. According to the fifth embodiment, a determining unit for determining the amount of movement (moving velocity) of the capsule is arranged, the vital information (here, image information) is efficiently obtained when the amount of movement is small by moving the capsule by the magnetic force.

FIG. 9 shows a capsule medical apparatus 1D according to the fifth embodiment.

The capsule medical apparatus 1D comprises: a capsule 3D which examines the coelom of the patient 2; an extracorporeal unit 4D which stores the image picked-up by the capsule 3D; a personal computer (PC) 5D which detects the position of the capsule 3D from the signal received by the extracorporeal unit 4D and controls the capsule so that it is moved when the change in position is small; the monitor 6 which displays the vital information, etc.; a magnetic field changing device 51 which changes the direction of the magnetic field under the control operation of the PC 5D; and a rotating magnetic field generating device 52 which generates rotating magnetic field by using the magnetic field changing device 51.

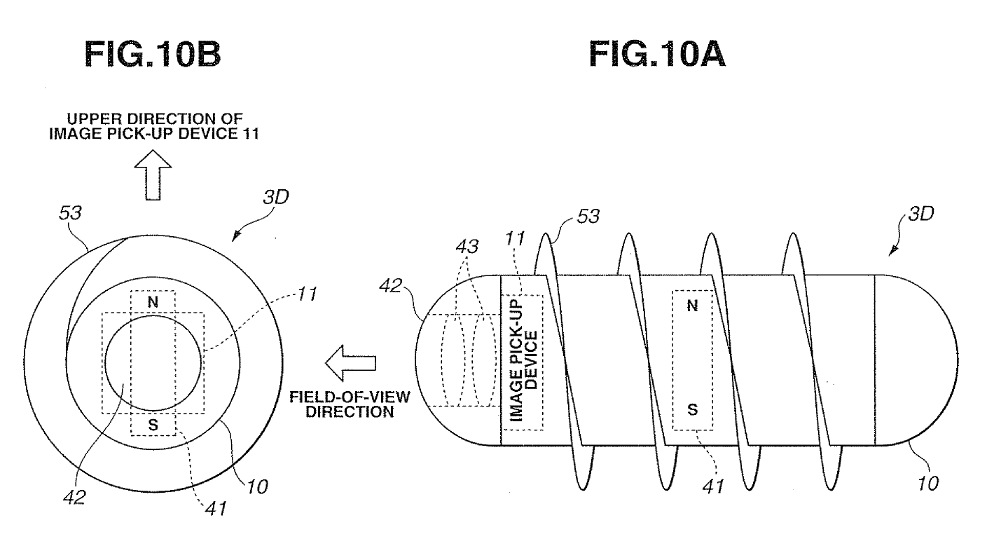

FIG. 10A is a side view of the capsule 3D and FIG. 10B is a front view of the capsule 3D in the field-of-view direction.

Referring to FIG. 10A, the capsule 3D comprises an operating-direction converting unit comprised of a screw portion 53 which rotates it by changing the direction of the magnet 41 in the capsule 3C shown in FIG. 8 to the direction perpendicular to the field-of-view direction and by applying the rotating magnetic field to the magnet 41 and which is formed by winding spiral projections to the outer surface of the capsule container 10 in the axial direction of the capsule 3D. When the capsule 3D inserted in the coelom is moved, the rotation of the capsule 3D enables the screw portion 53 to drive the capsule 3D.

Specifically, the objective lens 43 is arranged in the observation window 42 so that the central axis of the capsule 3D matches the optical axis of the objective lens 43. The center of the image pick-up surface of the image pick-up device 11 is located on the optical axis of the objective lens 43. The post-shaped magnet 41 having the circular- or quadrangular-shaped cross section is arranged in the capsule 3D so that its central axis in the longitudinal direction passes through the central axis of the capsule 3D and is perpendicular to the central axis of the capsule 3D, specifically, in the upper direction of the image pick-up device 11 as shown in FIG. 10B.

That is, the magnet 41 is arranged in the capsule 3D so that the magnetic directions of the N and S poles thereof are in a specific direction (upper direction in this case) on the image pick-up surface of the image pick-up device 11. Thus, it is recognized in which direction the current specific direction (upper direction) of the image of the capsule 3D is based on the direction where the outer magnetic field is detected by the magnet 41.

The extracorporeal unit 4D shown in FIG. 9 is connected to the antenna array 21 comprising a plurality of antennas and comprises a receiving circuit 54 being connected to the antenna array 21 and for receiving signal from the capsule 3D, the image storing device 26 for storing the image, and the RTC 27.

The signal received by the receiving circuit 54 is transmitted to the image storing device 26 and the image information is stored with the date data from the RTC 27 as shown in FIG. 7.

The data on the signal intensity received by the receiving circuit 54 by switching the antenna is transmitted to the PC 5D.

According to the fifth embodiment, the capsule 3D is set to the image pick-up operation upon the insertion in the coelom (the image pick-up operation is externally controlled according to a seventh embodiment).

The PC 5D comprises: the position detecting circuit 24 which detects the position of the capsule 3D from the data on the signal intensity outputted from the receiving circuit 54; a control circuit 57 having a moving-amount detecting function 56 for detecting the amount of movement on time series based on the detected positional data; and the image display processing circuit 29 for the image display processing.

When it is determined that the amount of movement detected by the moving-amount detecting function 56 is small, the control circuit 57 transmits the control signal to the magnetic field changing device 51, the magnetic field changing device 51 starts the operation by the control signal, the rotating magnetic field generating device 52 generates the rotating magnetic field, the rotating magnetic field is applied to the magnet 41 of the capsule 3D for the rotation, and the capsule 3D is driven.

The magnetic field generating device 52 comprises: an amplifier which amplifies a driving signal from the magnetic field changing device 51; and a three-axial magnet which is set to freely change the magnetic field in the three-axial direction and which generates the rotating magnetic field by the rotation as a result of receiving the driving signal amplified by the amplifier.

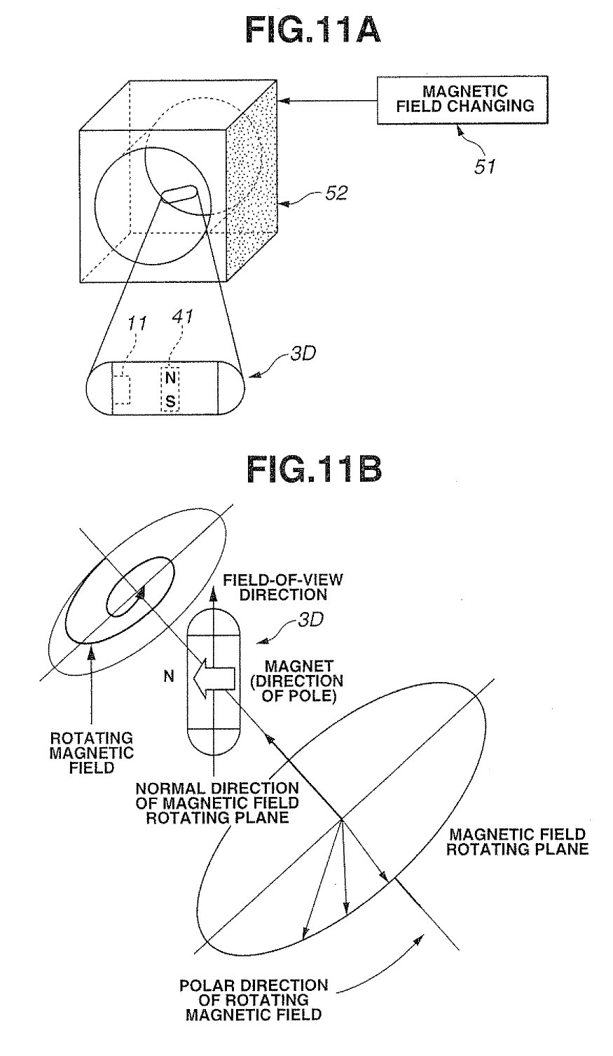

FIG. 11A shows the structure of the rotating magnetic field generating device 52, and FIG. 11B shows an explanatory diagram of the operation affected to the magnet 41 in the capsule 3D by the rotating magnetic field.

Referring to FIG. 11A, the magnetic field generating device 52 has a hollow portion so that it is arranged around the patient who swallows the capsule 3D.

Referring to FIG. 11B, the direction of the rotating magnetic field is sequentially changed rotatably on the magnetic filed rotating plane by applying the rotating magnetic field to the capsule 3D. Thus, the rotating magnetic force acts to the magnet 41 and the capsule 3D including the magnet 41 is rotated.

In accordance with the rotation of the rotating magnetic field, (although it is first detached), the rotating plane of the rotating magnetic field matches the rotating plane of the magnet 41 in the capsule 3D.

The moving-amount detecting function 56 detects the reducing state and stopping state of the moving velocity in the coelom of the patient 2 in the capsule 3D. Further, the capsule 3D is rotated, thereby rotating the screw portion 53 arranged to the outer peripheral surface of the capsule 3D. As a result, the capsule 3D is efficiently driven in the coelom.

The driving of the capsule 3D enables efficient acquisition of the image information in the coelom.

The position detecting circuit 24 stores the detected positional data as the positional information containing the date data, and outputs the stored information to the image display processing circuit 29. The image information is displayed together with the positional information.

When it is determined that the amount of movement is small, the control circuit 57 controls the image storing device 26 so that the image is decimated and stored.

The control circuit 57 may transfer to the image storing device 26, the positional data detected by the image detecting circuit 24, and the image storing device 26 may store the image information together with the positional information containing the date data from the RTC 27.

With the above-described structure and operation according to the fifth embodiment, the amount of movement of the capsule 3D is small, then, the control operation is performed so that the driving force for forced movement is externally applied to the capsule 3D. Accordingly, when the amount of movement is reduced, it is increased and the vital information in the coelom is efficiently obtained.

### Sixth Embodiment

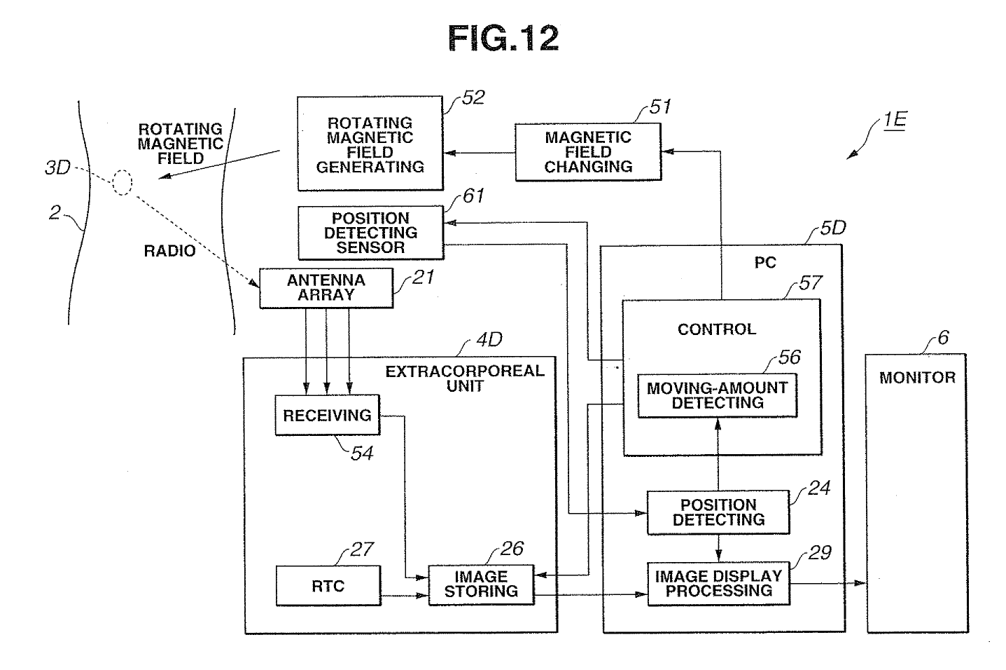

Next, a sixth embodiment of the present invention will be described with reference to FIG. 12. FIG. 12 shows a capsule medical apparatus 1E according to the sixth embodiment.

Referring back to FIG. 9, the position of the capsule 3D is detected based on the signal intensity which is obtained by switching the antenna and by receiving the signal in the capsule medical apparatus 1D. However, according to the sixth embodiment, arranged around the patient 2 is a position detecting sensor 61 using the two three-dimensional magnetic sensor units disclosed in Japanese Unexamined Patent Application Publication No. 2000-179700. Further, the signal output as the result of detecting the magnetic field of the magnet 41 by the position detecting sensor 61 is inputted to the position detecting circuit 24, and the position of the capsule 3D is detected (from the position of the magnet 41).

That is, the capsule medical apparatus 1E is formed by further adding the position detecting sensor 61 to the capsule medical apparatus 1D shown in FIG. 9. Further, the output from the position detecting sensor 61 is inputted to the position detecting circuit 24 in a personal computer 5D.

In this case, in order to detect the position of the capsule 3D without the influence of the rotating magnetic field upon setting the rotating magnetic field generating device 52 to the operating state, the control circuit 57 controls the operation of the position detecting sensor 61 and further controls the magnetic field changing device 51 so as to prevent the matching of a timing for detecting the position to a timing for applying the rotating magnetic field.

The above-mentioned control operation realizes the position detection with high accuracy.

Other structures are the same as those according to the fifth embodiment, and the operations are the same as those according to the fifth embodiment, excluding the point that the position of the capsule is detected by the magnetic field in place of the signal intensity transmitted by the electric waves according to the fifth embodiment.

According to the sixth embodiment, the same advantages as those according to the fifth embodiment are obtained. Advantageously, the magnetic field changing device 51 is used only when necessary because the portion for obtaining the image is formed independently of the portion for detecting the position and for controlling to apply the magnetic field.

### Seventh Embodiment

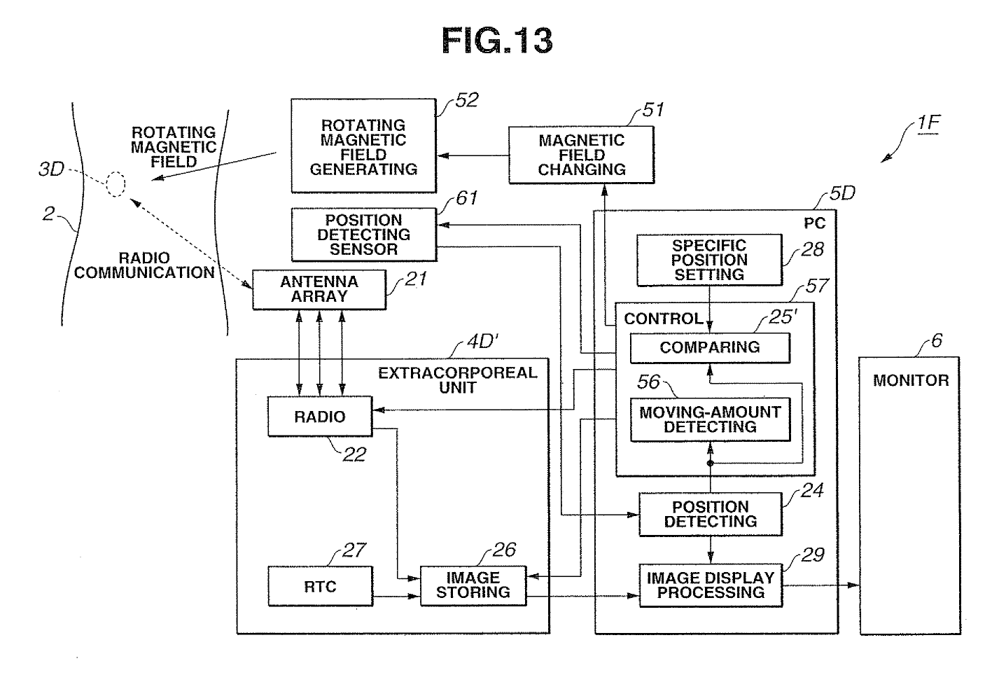

Next, a seventh embodiment of the present invention will be described with reference to FIG. 13. FIG. 13 shows a capsule medical apparatus 1F according to the seventh embodiment.

The capsule medical apparatus 1F is formed by adding the position setting unit 28 (described above according to the first embodiment) to the PC 5D in the capsule medical apparatus 1E shown in FIG. 12, and the control circuit 57 receives the positional data for starting and ending the image pick-up operation of the position setting unit 28, previously before the capsule 3 is inserted (or swallowed) in vivo.

The control circuit 57 comprises a comparing function 25′ (of the comparing circuit 25) which determines whether or not the positional data detected by the position detecting circuit 24 matches, e.g., the positional data for starting the image pick-up operation. Further, the control circuit 57 transmits to the capsule 3D via the radio circuit 22 of the extracorporeal unit 4D′, the control signal which starts or ends the image pick-up operation based on a result of the comparing function 25′ (the extracorporeal unit 4D′ in this case uses the radio circuit 22 for receiving and transmitting the signal in the extracorporeal unit 4D shown in FIG. 12, in place of the receiving circuit 54).

Specifically, in the examination of the small intestine, for example, the position setting unit 28 sets a first space and a second space near the duodenum and the appendix, respectively, for two positions (areas) for starting and ending the image pick-up operation.

Further, the control circuit 57 in the PC 5D controls the operation of the magnetic field changing device 51 and the rotating magnetic field generating device 52 so that the capsule 3D is early led to the first space until the capsule 3D which is swallowed reaches the first space. In this state, the image is not obtained.

The capsule 3D reaches the first space, then, the control circuit 57 recognizes (by using the comparing function 25′) that the capsule 3D reaches the first space, and it instructs the start for capturing the image by the capsule 3D and the stop of the rotating magnetic field to the magnetic field changing device 51 via the radio circuit 22 in the extracorporeal unit 4D′.

The capsule 3D captures the image and moves in the small intestine. After that, the capsule 3D passes through the small intestine and reaches the appendix, thus satisfying a condition indicating that the capsule 3D exists in the second space. The control circuit 57 detects this state (by using the comparing function 25′), and allows the capsule 3D to stop capturing the image. Further, the control circuit 57 controls so that the rotating magnetic field is generated again and the capsule 3D early passes through the large intestine.

Accordingly, the observer confirms the image of only the target portion (the small intestine, in this case), and the observation becomes efficient. Advantageously, the patient 2 reduces the examining time.

### Eighth Embodiment

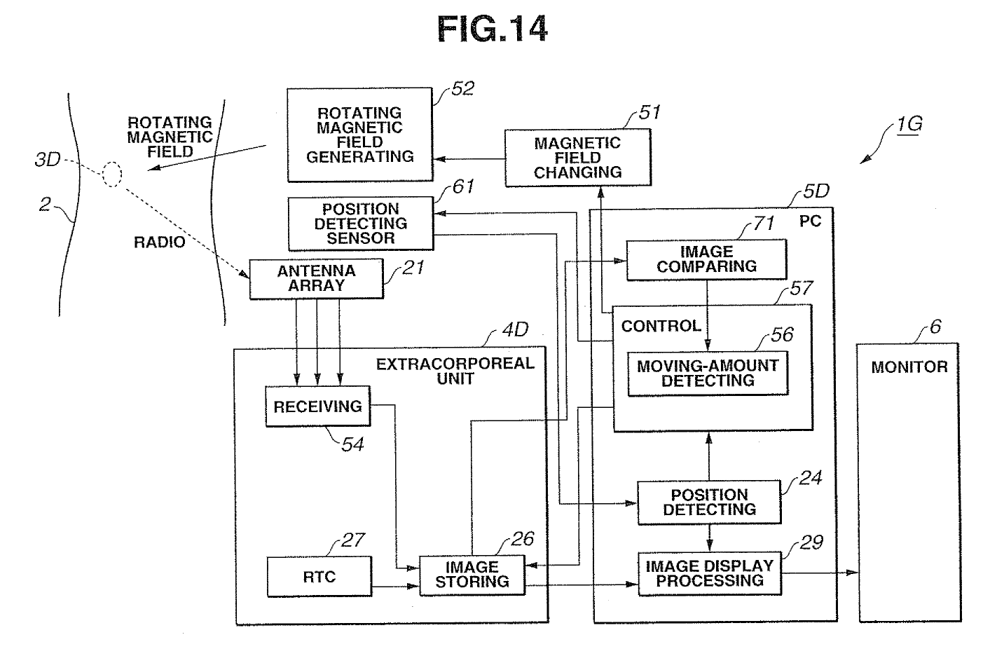

Next, an eighth embodiment of the present invention will be described with reference to FIG. 14. FIG. 14 shows a capsule medical apparatus 1G according to the eighth embodiment.

The capsule medical apparatus 1G is formed by arranging an image comparing unit 71 to the PC 5D in the capsule medical apparatus 1E shown in FIG. 12 so as to detect the amount of movement of the capsule 3D based on the image data outputted on time series received by the extracorporeal unit 4D.

In the capsule medical apparatus 1E shown in FIG. 12, the position detecting circuit 24 generates the positional data of the capsule 3D based on the output signal from the output detecting sensor 61, the positional data is inputted to the moving-amount detecting function 56, and the amount of movement of the capsule 3D is detected. However, the image data outputted via the image storing device 26 is inputted to the image comparing unit 71 arranged to the PC 5D, the amount of change in image is detected by processing for detecting the amount of image correlation of a plurality of pieces of the image data, and the detection result is inputted to the moving-amount detecting function 56, thus detecting the amount of movement of the capsule 3D, according to the eighth embodiment.

The image data compared by the image comparing unit 71 may use the data from the image storing device 26, it may be inputted to the image comparing unit 71 via the image display processing circuit 29, or it may use the data of the output signal from the receiving circuit 54.

As mentioned above, according to the eighth embodiment, the plurality of images on time series are subjected to the correlation processing, and the change in image is detected. When the image is not changed, the rotating magnetic field is applied to the capsule 3D via the control circuit 56 and the capsule 3D is forcedly moved. Accordingly, the capsule 3D is moved. The time for observing the same portion or substantially the same portion is reduced and the examination becomes efficient.

### Ninth Embodiment

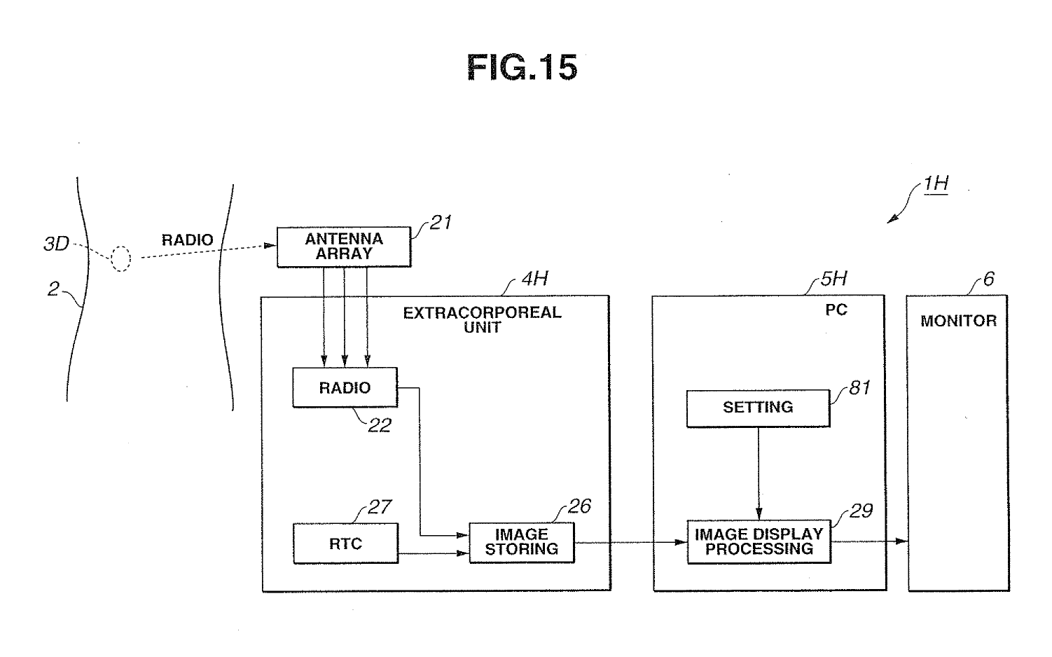

Next, a ninth embodiment of the present invention will be described with reference to FIG. 15. FIG. 15 shows a capsule medical apparatus 1H according to the ninth embodiment.

The positions for starting and ending the image pick-up operation are designated and the image pick-up operation is performed between the positions as shown in FIG. 5 according to the second embodiment. However, in the capsule medical apparatus 1H according to the ninth embodiment, the image information stored in an extracorporeal unit 4H is captured in a PC 5H and is displayed on the monitor 6, first and second specific picked-up images may be designated to the image displayed on the monitor 6 as a portion for starting the observation and a portion for ending it by using a setting unit 81 such as a keyboard arranged to the PC 5H.

As a result of the designation, the time bar is displayed in the positional data display area A3 by assuming that the time for capturing the first specific picked-up image is the start point and the time for capturing the second specific picked-up image is the end point, as mentioned above according to the modification of the second embodiment.

The capsule medical apparatus 1H is formed by modifying the capsule medical apparatus according to the second embodiment. That is, in the capsule medical apparatus 1H, the extracorporeal unit 4 comprises: the radio circuit 22 connected to the antenna array 21 (or receiving circuit); the image storing device 26; and the RTC 27, and the PC 5H comprises the image display processing unit 29 and the setting unit 81.

According to the ninth embodiment, the image display processing unit 29 in the PC 5H captures the image information stored in the image storing device 26 in the extracorporeal unit 4H and the picked-up image is displayed on the monitor 6.

As mentioned above, the operator designates the first specific picked-up image and the second specific picked-up image as both ends of the observing range by using the setting unit 81. Then, the picked-up image is displayed between both ends of the observing range, and it is displayed which position percent of the picked-up image to the observing range as 100 percent.

In other words, according to the ninth embodiment, the display operation is similar to the display example shown in FIG. 5 according to the second embodiment. According to the second embodiment, the first and second specific picked-up images are not designated but the spatial positions (areas) are displayed. However, according to the ninth embodiment, the specific picked-up images at both ends of the observing range are designated, the picked-up image is displayed for the time period between the time for capturing the specific picked-up images, and it is displayed which percent on time base of the image pick-up time for capturing the image to the time period between the time for capturing the specific picked-up images.

Therefore, even when the position detecting unit is not arranged to the capsule 3 according to the ninth embodiment, advantageously, the position of the picked-up image is easily grasped within the observing range.

### Tenth Embodiment

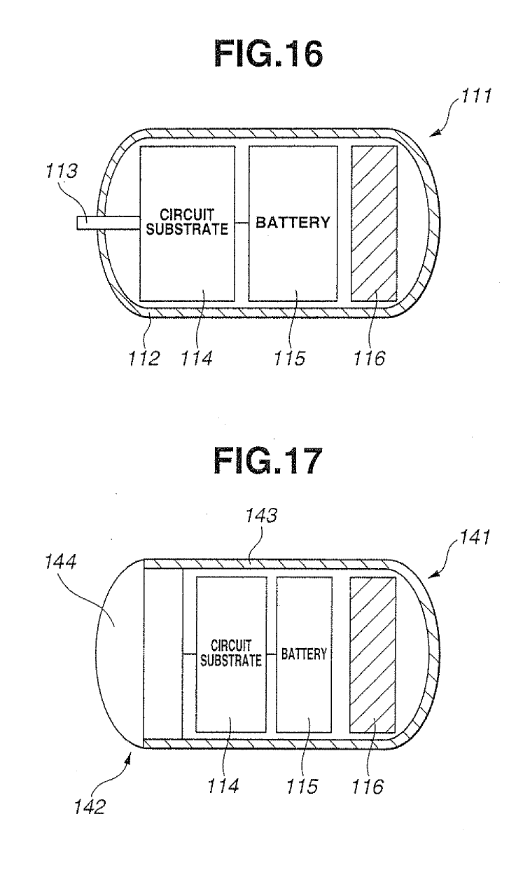

Next, a tenth embodiment of the present invention will be described with reference to FIG. 16. The structure according to the tenth embodiment is similar to that according to the first embodiment, and an unnecessary portion is not described. FIG. 16 shows a capsule 111 according to the tenth embodiment of the present invention. According to the tenth embodiment, a pH sensor 113 is provided in place of the image pick-up device 11 and the illumination device 12.

The capsule 111 includes a capsule main body 112 which forms with watertightness comprising a cylindrical portion and a cover for roundly covering both ends thereof. A detecting unit of the pH sensor 113 for detecting pH is provided (or exposed) at one end portion of the capsule main body 112.

When the detecting unit of the pH sensor 113 is projected from a hole portion of the capsule main body (container) 112, the capsule main body 112 is watertight by fixing with an adhesive having a high watertight function.

A rear end side of the pH sensor 113 is connected to a circuit substrate 114 having a function of processing for pH detection and communication means for storing and externally transmitting the pH data, arranged in the capsule main body 112. The circuit substrate 114 is connected to a battery 115 which supplies power for operating the circuit substrate 114. The battery 115 uses silver oxide or a fuel battery which has a high degree of freedom in shape with high efficiency.

According to the tenth embodiment, the capsule main body 112 accommodates a permanent magnet or a magnetic member 116 near an end portion on the opposed side of the pH sensor 113.

For example, the collection is executed by using an elongated-tube-shaped collecting tool for housing the permanent magnet near the edge thereof, such as an ileus tube, when the capsule 111 is lodged at a narrow portion.

The operation with the above structure will be described according to the tenth embodiment with reference to a flowchart shown in FIG. 2. A description is given by replacing the image pick-up operation, the image data, and the image storing device 26 with the pH estimation, the pH data, and a storing device, respectively.

In step S1, the PC 5 sets the positions previously before the capsule 111 is inserted (or swallowed) in vivo. That is, the position setting means 28 sets both of positions for starting and ending the pH estimation, and transmits the positional data to the extracorporeal unit 4. Specifically, when the small intestine is examined, the position of the duodenum is set as the position for starting the pH estimation, and the appendix is set as the position for ending the pH estimation.

In this case, upon designating the position for starting the pH estimation, a plurality of positions are designated as positions for starting the pH estimation so as to designate an area near the duodenum, and the comparing circuit 25 may determine whether or not the current position is the position for starting the pH estimation depending on whether or not it is within the range of the plurality of positions.

After ending the setting of the position for starting or ending the pH estimation, in step S2, the positional setting data is transmitted to the extracorporeal unit 4, and the extracorporeal unit 4 stores the setting data to the memory in the comparing circuit 25. In step S3, the PC 5 is detached from the extracorporeal unit 4 and the patient 2 swallows the turned-on capsule 111.

In step S4, the capsule 111 transmits the signal for detecting the position. The extracorporeal unit 4 switches a plurality of antennas forming the antenna array 21, demodulates the signal for detecting the position by the radio circuit 22, and sends the demodulated signal to the position detecting circuit 24.

In step S5, the position detecting circuit 24 calculates the position of the capsule 111 and transmits the calculated positional data to the comparing circuit 25.

In step S6, the comparing circuit 25 determines whether or not the calculated positional data matches (overlaps to) the positional data on the position for starting the pH estimation in the position setting data within the threshold value. When NO in step S6, the processing routine returns to step S4 whereupon the position is calculated from the signal for detecting the position transmitted from the capsule 111 and the processing for matching to the positional data on the position for starting the pH estimation.

When the capsule 111 reaches the position for starting the pH estimation, the calculated positional data matches, within the threshold value, the setting data which is set as the position for starting the pH estimation stored in the comparing circuit 25. In step S7, the comparing circuit 25 in the extracorporeal unit 4 transmits the matching result to the control circuit 23. The control circuit 23 transmits the signal for instructing the start of the pH estimation to the capsule 111 via the radio circuit 22.

On the capsule 111 side, the control circuit 13 previously stores the instruction contents of the signal for instructing the start of the pH estimation and reads the contents by comparing it with storing means of an instructing code. In step S8, the pH estimation is started and the data on the pH estimation and the positional data are transmitted simultaneously. In this case, the pH sensor 113 is driven for a predetermined period.

The pH estimation is not performed until the capsule 111 reaches the position for starting the pH estimation and therefore the energy consumption of the battery 16 is saved.

On the extracorporeal unit 4 side, the pH data received via the radio circuit 22 is inputted to the storing device 26. In step S9, the storing device 26 stores the pH data and further stores the positional data detected by the position detecting circuit 24 and the date data from the RTC 27.

Referring back to 3A, the pH information is sequentially stored in the storing device 26 in order of the header, pH data, positional data, and footer. As mentioned above, the capsule 111 reaches the position for starting the pH estimation and, then, the capsule 111 starts the pH estimation. Further, the capsule 111 sequentially transmits the data on the pH estimation and the positional signal to the extracorporeal unit 4. The extracorporeal unit 4 stores the pH data, positional data, and date data with a correlation thereamong.

The positional data detected by the position detecting circuit 24 is transmitted to the comparing circuit 25. In step S10, the comparing circuit 25 determines whether or not the positional data transmitted from the position detecting circuit 24 matches the positional data at the position for ending the pH estimation within the threshold value. When NO in step S10, the processing routine returns to step S8 whereupon the pH estimation continues.

The capsule 111 reaches the position for ending the pH estimation and then such a fact is detected by matching the positional data to the setting data stored in the memory in the comparing circuit 25. The detecting result is transmitted to the control circuit 23.

In step S11, the control circuit 23 transmits the signal for instructing the ending of the pH estimation to the capsule 111. In step S12, the capsule 111 receives the transmitted signal and then the pH estimation stops.

According to the tenth embodiment, the pH sensor 113 for detecting pH is used as (medical) vital information detecting means. In addition, a temperature sensor, pressure sensor, optical sensor or blood sensor (specifically, sensor for detecting hemoglobin) may be used as the vital information detecting means. Receiving and transmitting method between the capsule 111 and the extracorporeal unit 5 are the same as that according to the first embodiment as mentioned above.

According to the tenth embodiment, a sensor unit (detecting unit) obtains information such as the chemical amount of the solution in vivo (pH), temperature of the organ, pressure from the luminal surface on the capsule outer-surface upon passage through the capsule, brightness in vivo, and the amount of hemoglobin of the organ (presence or absence of the bleeding). The obtained data is transmitted to receiving means in the extracorporeal unit extracorporeally arranged, by radio communication means in the capsule.

The data obtained by the receiving means is stored and is compared with a reference value. Thus, the abnormal state such as the disease and hemorrhage, the position upon passage through the capsule, and the passage state are determined on the outside of the body by a doctor and a health care worker such as a co-medical.

Advantageously, the capsule 111 estimates the pH in the gastrointestinal tract and the amount of hemoglobin and the diagnosis of disorder in the gastrointestinal tract and the physiological analysis are performed without pain of a subject. A plurality of sensors are provided to fit each purpose and the examination is efficiently performed.

The examination data is transmitted and received only for an estimation period and therefore the transmission and reception are efficiently performed. Since the sensor operation period is only the period for the estimation, advantageously, the battery life is extended and the estimation for a long time is possible. Unnecessary data is reduced upon confirming the data to record the data only for the estimation period, advantageously, the examination is smoothly performed.

Although the capsule 111 has the sensors as shown in FIG. 16, a capsule 141 having an ultrasonic probe 142 may be used as shown in FIG. 17, in place of the sensors shown in FIG. 16.

An acoustic lens 144 arranged to the front surface of the ultrasonic probe 142 is exposed to the outer surface of a capsule main body 143 in front of the capsule main body 143 in the capsule 141. The acoustic lens 144 is fixed to the capsule main body 143 by an adhesive in a watertight fashion and the capsule prevents the water from penetrating into the inside thereof.

In the capsule on the back side of the ultrasonic probe 142, an ultrasonic receiving and transmitting circuit and the circuit substrate 114 for generating an ultrasonic tomogram based on a signal therefrom are arranged. The circuit substrate 114 is driven by power from the battery 115. On the rear end side, the permanent 116 is housed.

In the capsule 141, the ultrasonic tomogram in the coelom is generated by the ultrasonic receiving and transmitting circuit formed on the circuit substrate 114. The captured data is transmitted to the extracorporeal receiving means, similarly the case shown in FIG. 16. Thus, the diagnosis about the abnormal state is performed for a long time in the depth direction of a deep portion in the coelom such as the small intestine.

Both the extracorporeal receiving means and optical observing means (image pick-up means) may be provided. With the above-mentioned structure, the diagnosis is executed for the surface in the coelom and the deep portion.

Other structures and operations are mentioned above and are not described. With the foregoing structure, advantageously, the ultrasonic probe 142 is operated for only the period of the passage through the estimation target. Thus, advantageously, the battery life is extended. Further, advantageously, the unnecessary data is reduced upon confirming the data to record the data only for the estimation target and the examination is executed smoothly.

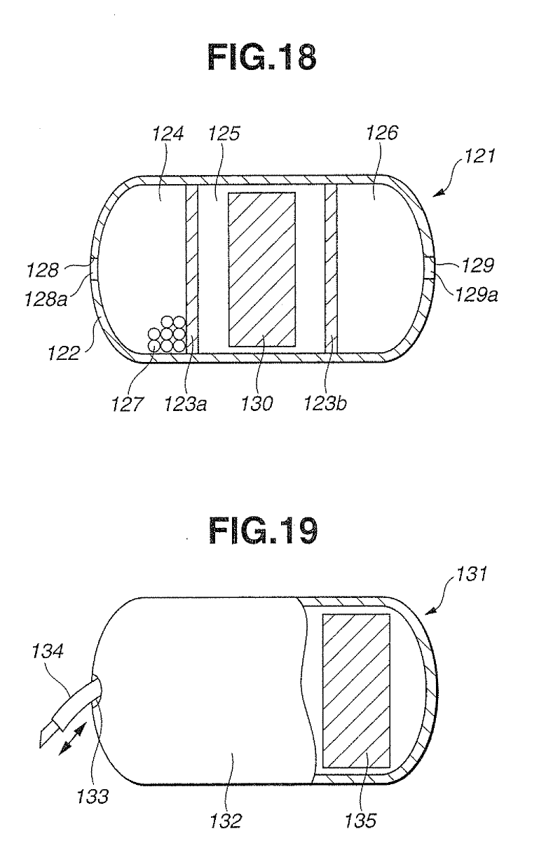

FIG. 18 shows a capsule 121 according to a second modification of the tenth embodiment.

In the capsule 121, a capsule main body 122 comprises a cylindrical portion and a cover for roundly covering both ends thereof. Further, the capsule main body 122 is partitioned by partitioning members 123a and 123b at two portions in the longitudinal direction. Further, the capsule 121 includes three containing means of a drug containing portion 124, a permanent magnet/magnetic member containing portion 125, and a body fluid portion 126.

The drug containing portion 124 contains a drug 127 for curing and further contains a drug slit 128 as opening means for discharging the contained drug 127 to the outside.

The body fluid portion 126 arranged on the opposite side of the drug containing portion 124 has a body fluid absorbing slit 129 for absorbing the body fluid from the outside of the capsule main body 122.

The permanent magnet/magnetic member containing portion 125 contains the permanent magnet or the magnetic member 130.

Electric valves 128a and 129a are arranged to the openings of the drug slit 128 and the body fluid absorbing slit 129, thereby controlling the opening and closing operations based on the control signal.

Next, the operation will be described.

First, the PC 5 sets the space in which to perform the medical activity, previously before the capsule 3 is inserted (or swallowed) in vivo. Here, a portion for discharging the drug is designated.

After ending the setting of the space for discharging the drug, spatial setting data is transmitted to the extracorporeal unit 4 and the extracorporeal unit 4 stores the setting data in the memory in the comparing circuit 25.

The PC 5 is detached from the extracorporeal unit 4 and the patient 2 swallows the turned-on capsule 121.

Then, the capsule 121 transmits the signal for detecting the position. The extracorporeal unit 4 switches a plurality of antennas forming the antenna array 21, demodulates the signal for detecting the position by the radio circuit 22, and sends the demodulated signal to the position detecting circuit 24.

The position detecting circuit 24 calculates the position of the capsule 121 and transmits the calculated positional data to the comparing circuit 25.

The comparing circuit 25 determines whether or not the calculated positional data matches (overlaps to) the spatial setting data. When no matching, the position is calculated from the signal for detecting the position transmitted from the capsule 121 and processing for matching the positional data of the capsule 121 to the spatial setting data is repeated.

When the capsule 121 reaches the space for discharging the drug (spatial setting data), the calculated positional data matches the setting data which is set as the position for discharging the drug stored in the memory of the comparing circuit 25, within the threshold value. In this case, the comparing circuit 25 in the extracorporeal unit 4 transmits the matching result to the control circuit 23. The control circuit 23 transmits a signal for instructing the start to discharge the drug to the capsule 121 via the radio circuit 22.

On the capsule 121 side, the control circuit 13 receives the signal for instructing the start to discharge the drug, and reads the contents by comparing it with a prestored instructing code. The drug starts to be discharged.

The electric valve 128a or 129a is opened. Thus, the drug 127 is administrated and the body fluid is absorbed. A discharging signal is transmitted from the extracorporeal unit and is received by the capsule 121, thereby controlling the discharging operation.

After that, the capsule 121 is detached from the discharging space of the drug (space stored in the spatial setting data), then, the output of the comparing circuit 25 is changed, and the data on the change is transmitted to the control circuit 23. The control circuit 23 transmits a signal for instructing the stop of discharge of the drug to the capsule 121 via the radio circuit 22. In the capsule 121, the control circuit 13 reads the instruction for stopping the discharge of the drug by comparing it with the prestored instruction code, and stops the discharge operation of the drug.

According to the second modification of the tenth embodiment, the body fluid is absorbed for the curing and examination only at the target portion. With the above structure, the drug is discharged only to the target portion. Or, the body fluid is collected only at the target portion. Advantageously, the medication and the examination are efficiently executed.

FIG. 19 shows a capsule 131 according to a third modification of the tenth embodiment.

In the capsule 131, a capsule main body 132 comprises a cylindrical portion and a cover for covering it at both ends thereof. An opening 133 is arranged on one end portion side of the capsule to freely project a needle for syringe 134 for injecting the drug. The capsule main body 132 further comprises driving means which freely projects the needle for syringe 134 and control means thereof. The extracorporeal unit transmits a control signal and the capsule 131 receives the control signal. Accordingly, the needle for syringe 134 is projected and the drug is injected. A permanent magnet or magnetic member 135 is housed near the end portion on the opposite side of an opening 133 in the capsule main body 132.

Next, the operation will be described.

The PC 5 sets the space previously before the capsule 131 is inserted (or swallowed) in vivo. Here, a portion for injecting the drug is designated.

After ending the setting of the space for injecting the drug, the spatial setting data is transmitted to the extracorporeal unit 4. The extracorporeal unit 4 stores the setting data in the memory in the comparing circuit 25.

The PC 5 is detached from the extracorporeal unit 4, and the patient 2 swallows the turned-on capsule 131.

Then, the capsule 131 transmits the signal for detecting the position. The extracorporeal unit 4 switches a plurality of antennas forming the antenna array 21, demodulates the signal for detecting the position by the radio circuit 22, and sends the demodulated signal to the position detecting circuit 24.

The position detecting circuit 24 calculates the position of the capsule 131 and transmits the calculated positional data to the comparing circuit 25.

The comparing circuit 25 determines whether or not the calculated positional data matches (overlaps to) the spatial setting data. When no matching, the position is calculated from the signal for detecting the position transmitted from the capsule 131 and the processing for matching the positional data to the spatial setting data is repeated.

When the capsule 131 reaches the space for discharging the drug (spatial setting data), the calculated positional data matches the setting data which is set as the position for discharging the drug stored in the memory of the comparing circuit 25, within the threshold value. In this case, the comparing circuit 25 in the extracorporeal unit 4 transmits the matching result to the control circuit 23. The control circuit 23 transmits the signal for instructing the discharge of the drug to the capsule 131 via the radio circuit 22.

On the capsule 131 side, the control circuit 13 receives a signal for instructing the start to inject the drug, and reads the contents by comparing it with a prestored instructing code. The operation for injecting the drug starts (operation for operating a driving unit for projecting the needle for syringe 134, projecting the needle for syringe, and injecting the drug starts).

Specifically, a homeostatic agent such as ethanol and dry chemical is injected to a bleeding portion and the bleeding stops.

According to the third modification, the battery life is extended and then the treatment such as the stop of bleeding is performed. With the above structure, the drug is injected only to the target portion. Advantageously, the drug is efficiently injected.

### Eleventh Embodiment

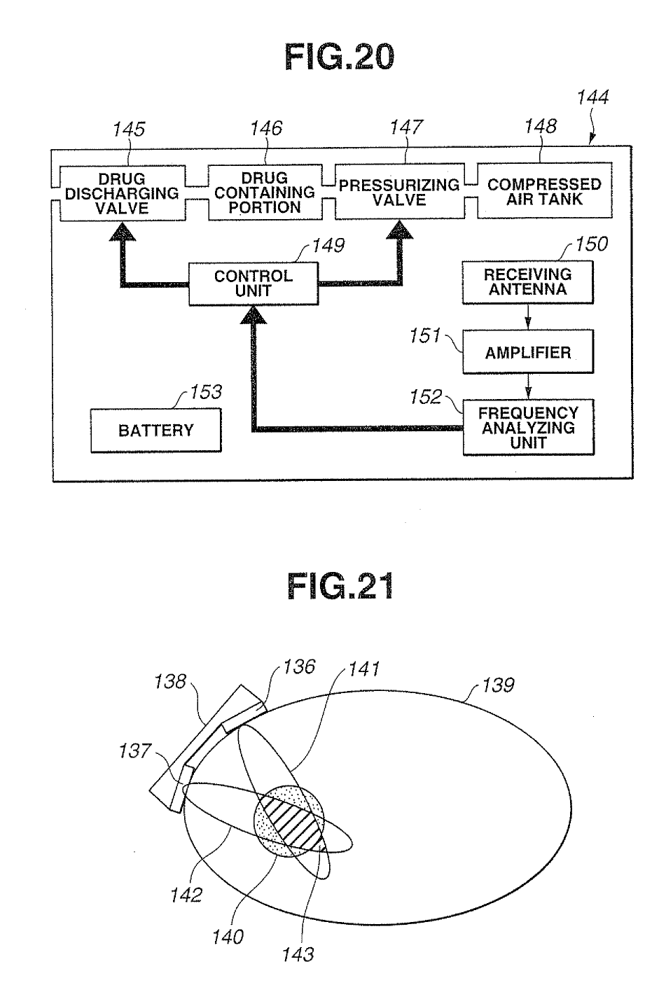

Next, an eleventh embodiment of the present invention will be described with reference to FIGS. 20 and 21. FIG. 20 shows a capsule 144 according to the eleventh embodiment. The capsule 144 includes a drug discharging valve 145 which is arranged to a pipe for discharging the drug contained in a drug containing portion 146 to the outside of the capsule 144, and which executes the opening and closing operation of the pipe.

Further, the capsule 144 has a compressed air tank 148 containing compressed air. The compressed air tank 148 is connected to the drug containing portion 146 by the pipe. A pressurizing valve 147 is arranged in the pipe to open and close the pipe between the compressed air tank 148 and the drug containing portion 146.

A receiving antenna 150 arranged to the capsule 144 receives a signal transmitted through a transmitting antenna 136 and a transmitting antenna 137 set to the body surface of a patient 139 shown in FIG. 21.

The signal received by the antenna 150 is amplified by an amplifier 151 and is transmitted to a frequency analyzing unit 152 for frequency analysis. An output from the frequency analyzing unit 152 is transmitted to a control unit 149. The control unit 149 opens and closes the pressurizing valve 147 and the drug discharging valve 145 based on the output result of the frequency analyzing unit 152. The capsule 144 has a battery 153 for supplying power to the drug discharging valve 145, pressurizing valve 147, amplifier 151, frequency analyzing unit 152, and control unit 149.

Referring to FIG. 21, reference numeral 136 denotes a first transmitting antenna for transmitting a signal having a frequency f1. The transmitting antenna 136 is an antenna having the directivity for sending electric waves with substantially elliptical-shaped intensity distribution as shown by reference numeral 141.

A second antenna 137 transmits a signal having a frequency f2. The transmitting antenna 137 is an antenna having the directivity for sending the electric waves with intensity distribution as shown by reference numeral 142.

The frequency f1 is different from the frequency f2. The first transmitting antenna 136 and the second transmitting antenna 137 are attached to a transmitting antenna base 138 at an angle (namely, the first and second transmitting antennas 136 and 137 do not exist on the same plane and crosses to each other in the body of a patient 139 as shown in FIG. 21). The angle may be adjusted.

The transmitting antenna base 138 is attached to the body surface of the patient 139. The transmitting antenna base 138 is fixed to the patient 139 by a band or tape (not shown). Referring to FIG. 21, reference numeral 140 denotes a spherical-shaped drug distributed target portion. The drug distributed target portion 140 is previously checked by a CT, an MRT, or an endoscope device and is designatedly positioned in the body.

Since the transmitting antenna 136 and the transmitting antenna 137 are attached at the angle, an area 143 shown by shading can transmit and receive the transmitting signals from the transmitting antenna 136 and the transmitting antenna 137.

Next, the operation will be described according to the eleventh embodiment.

The drug distributed target portion 140 is checked by the CT, the MRT, or the endoscope device and is previously designatedly positioned in the body. The transmitting antenna base 138 is attached to the body surface of the patient and is fixed by using a tape so that an area of the drug distributed target portion 140 is overlapped to the area 143. After that, a switch (not shown) is operated so that the transmitting antenna 136 and the transmitting antenna 137 start the transmission, thus starting the transmission.

Sequentially, the compressed air is contained in the compressed air tank 148, a drug (not shown) for distribution is contained in the drug containing portion 146. The operation of the capsule 144 is started by switching on a switch (not shown) of the closed capsule 144 in which both the pressurizing valve 147 and the drug discharging valve 145 are closed.

The patient 139 swallows the operated capsule 144.

The capsule 144 estimates receiving intensities from the transmitting antennas 136 and 137 by the receiving antenna 150 and moves.

When the capsule 144 does not exist in the area 143, the intensity of the frequency f1 does not reach a predetermined value, the intensity of the frequency f2 does not reach a predetermined value, or neither the frequency f1 nor the frequency f2 reaches a predetermined value. In this case, the control unit 149 continues this state. The information on this state is received by the receiving antenna 150, the frequency is amplified by the amplifier 151, the frequency information is analyzed by the frequency analyzing unit 152, and the analyzing result is transmitted to the control unit 149.

The capsule 144 reaches the area 143 and, then, both of the frequencies f1 and f2 are received with the intensity over the predetermined value. The information is received by the receiving antenna 150, the frequency is amplified by the amplifier 151, the frequency information is analyzed by the frequency analyzing unit 152, and is transmitted to the control unit 149.

The control unit 149 recognizes that the capsule 144 enters the area 143, and opens the pressurizing valve 147 and the drug discharging valve 145. Then, the compressed air in the compressed air tank 148 presses the drug in the drug containing portion 146 to the outside of the capsule 144. Accordingly, the drug can be distributed to the drug distributed target portion 140.

With the foregoing structure according to the eleventh embodiment, advantageously, the capsule 144 easily distributes the drug to the target portion and the amount of drug is reduced.

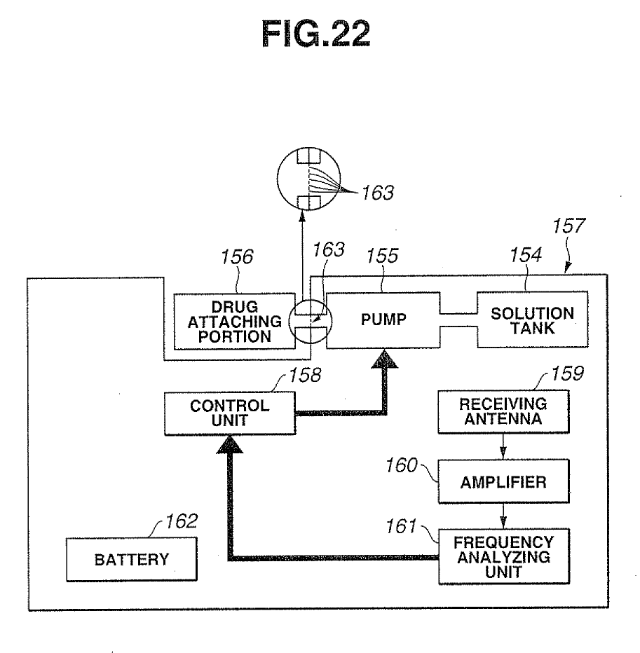

FIG. 22 shows a modification of the eleventh embodiment.

According to the modification, a drug attaching portion 156 is arranged to a surface of a capsule 157 in place of the drug containing portion 146. The drug attaching portion 156 has a minute hole 163 (shown in an enlarged view) connected to a pump 155 through a pipe. The pump 155 is connected to a solution tank 154 at one end thereof.

A drug is fixed to the drug attaching portion 156 by distribution onto fat. The solution tank 154 contains alcohol. The pump 155 is on/off-controlled by a control unit 158.

A receiving antenna 159, an amplifier 160, a frequency analyzing unit 161, and a battery 162 arranged to the capsule 157 have the same structure and functions as those of the capsule 144 shown in FIG. 20 and therefore they are not described.

The control unit 158 sends the alcohol from the solution tank 154 to the medicine attaching portion 156 through the minute hole 163 by switching on the pump 155. The alcohol solves the fat. Thus, the drug is discharged. Other operations and advantages are the same as those of the capsule 144 shown in FIG. 20.

In the above description, the screw portion 53 which is formed by, for example, winding spiral projections on the outer surface of the capsule container 10 in the axial direction of the capsule 3D refers to the operating-direction converting unit provided to the capsule. By having the protruding operating-direction converting unit as the screw portion formed by winding spiral projections, and the magnet, the capsule 3D is rotationally driven, that is, advances or retreats in the axial direction of the capsule 3D. The capsule 3D is driven using propulsion force generated by the rotation of the screw portion 53, not mere attraction force and repulsion force by the magnet, and therefore has a strong driving force. Moreover, because the capsule container 10 itself rotates, the capsule 3D is hardly effected by friction of the coelom wall or the like and can easily move to a desired direction even in a narrow coelom.

According to the present invention, the capsule medical apparatus comprising the capsule having medical treatment portions (e.g., the vital information detecting unit for obtaining vital information, the curing portion, the treatment portion, the organ removal portion, and a drug discharging portion), inserted or swallowed in vivo and an extracorporeally-arranged extracorporeal unit, comprises: the recognizing unit which recognizes whether or not the capsule exits in a space; the space setting unit which designates the space in vivo previously before the capsule is inserted (or swallowed) in vivo; and the control unit which controls a state of the capsule by an output signal from the recognizing unit. Thus, the operation for the medical activity only in the space or the operation for starting or stopping thereof is automatically controlled by previously designating the space for the operation and therefore the medical activity is efficiently performed.

In other words, according to the present invention, the capsule medical apparatus comprising the capsule having medical treatment portions, inserted or swallowed in vivo and an extracorporeally-arranged extracorporeal unit, comprises: the recognizing unit which recognizes whether or not the capsule exits in a space; the space setting unit which designates the space in vivo previously before the capsule is inserted (or swallowed) in vivo; and the control unit which controls a state of the capsule by an output signal from the recognizing unit. Accordingly, the operation for the medical activity only in the space or the operation for starting or stopping thereof is automatically controlled by previously designating the space for the operation and therefore the medical activity is efficiently performed.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A capsule medical apparatus comprising:
a capsule which is inserted or swallowed in vivo;
a space setting unit which designates a space in vivo previously before the capsule is inserted or swallowed in vivo;
a recognizing unit which recognizes whether or not the capsule exists in the space set by the space setting unit; and
a control unit which controls a state of the capsule by an output from the recognizing unit.

2. A capsule medical apparatus according to claim 1, wherein the recognizing unit is arranged in the capsule.

3. A capsule medical apparatus according to claim 1, further comprising:
an extracorporeal unit having a communication unit for communication with the capsule,
wherein the recognizing unit is arranged to the extracorporeal unit.

4. A capsule medical apparatus according to claim 1, wherein the space setting unit is arranged so that it comes into contact with the outer surface of the living body.

5. A capsule medical apparatus according to claim 1, wherein the capsule has a vital information obtaining unit which obtains vital information, and
the operation of the vital information obtaining unit is controlled based on the output from the recognizing unit by the control unit.

6. A capsule medical apparatus according to claim 1, wherein the capsule has a drug containing portion and a discharging portion which discharges the drug contained in the drug containing portion to the outside of the capsule medical apparatus, and
the operation of the discharging portion is controlled based on the output from the recognizing unit by the control unit.

7. A capsule medical apparatus according to claim 1, wherein the capsule has a drug attaching portion and a discharging control unit which controls the discharging operation of a drug attached to the drug attaching portion, and
the operation of the discharging control unit is controlled based on the output from the recognizing unit by the control unit.

8. A capsule medical apparatus according to claim 1, wherein the capsule has a removing unit which removes a material outside of the capsule in the capsule, and
the operation of the removing unit is controlled based on the output from the recognizing unit by the control unit.

9. A capsule medical apparatus according to claim 1, wherein the capsule has a treatment unit which treats the living body, and
the operation of the treatment unit is controlled based on the output from the recognizing unit by the control unit.

10. A capsule medical apparatus comprising a capsule having a vital information detecting unit for obtaining vital information, inserted in vivo, and an extracorporeal unit arranged in vitro, the capsule medical apparatus comprising:
a position detecting unit which detects a position of the capsule;
a space setting unit which designates a space in vivo previously before the capsule medical apparatus is inserted in vivo;
a comparing unit which compares information on the capsule position from the position detecting unit with the space set by the space setting unit and outputs a signal corresponding to a comparison result; and
a control unit which controls a state of the capsule based on a signal output from the comparing unit.

11. A capsule medical apparatus according to claim 10, wherein the control unit controls the operation of the vital information detecting unit based on the signal output from the comparing unit so that it starts.

12. A capsule medical apparatus according to claim 10, wherein the control unit controls the operation of the vital information detecting unit based on the signal output from the comparing unit so that it stops.

13. A capsule medical apparatus according to claim 10, wherein the control unit controls the operation for power management of the capsule based on the signal output from the comparing unit.

14. A capsule medical apparatus comprising a capsule having a discharging or absorbing unit for discharging a contained material of the capsule including a drug or for absorbing the vital substance such as the body fluid only at a target portion in vivo, inserted (or swallowed) in vivo, and an extracorporeal unit arranged in vitro, the capsule medical apparatus comprising:
a position detecting unit which detects a position of the capsule;
a space setting unit which designates a space in vivo previously before the capsule medical apparatus is inserted or swallowed in vivo;
a comparing unit which compares information on the capsule position from the position detecting unit with the space set by the space setting unit and outputs a signal corresponding to a comparison result; and
a control unit which controls the operation of the discharging or absorbing unit of the capsule, based on a signal output from the comparing unit.

15. A capsule medical apparatus comprising a capsule having a treatment unit for curing or treatment only at a target portion in vivo, inserted (or swallowed) in vivo, and an extracorporeal unit arranged in vitro, the capsule medical apparatus comprising:
a position detecting unit which detects a position of the capsule;
a space setting unit which designates a space in vivo previously before the capsule medical apparatus is inserted or swallowed in vivo;
a comparing unit which compares information on the capsule position from the position detecting unit with the space set by the space setting unit and outputs a signal corresponding to a comparison result; and
a control unit which controls the operation of the treatment unit of the capsule, based on the signal output from the comparing unit.

16. A capsule medical apparatus comprising a capsule for medical activity including examination, curing, or treatment only at a target portion in vivo, inserted or swallowed in vivo, and an extracorporeal unit arranged in vitro, the capsule medical apparatus comprising:
a position detecting unit which detects a position of the capsule; and
a space setting unit which designates a space in vivo previously before the capsule medical apparatus is inserted or swallowed in vivo,
wherein at least one of the position detecting unit and the space designating unit is substantially touched to the living body.

17. A capsule medical apparatus comprising a capsule having a vital information detecting unit for obtaining vital information, inserted or swallowed in vivo, and an extracorporeal unit arranged in vitro, the capsule medical apparatus comprising:
a display unit which sets a first portion and a second portion of the organ and displays observation information between the first and second portions at a ratio of time division; and
a capsule position detecting unit,
wherein the first portion is a portion where a first space set by a first space setting unit for setting a space previously before the capsule medical apparatus is inserted or swallowed in vivo, is overlapped to information on the capsule position from the position detecting unit, and
the second portion is a portion where a second space set by a second space setting unit for setting the space previously before the capsule medical apparatus is inserted or swallowed in vivo, is overlapped to the information on the capsule position from the position detecting unit.

18. A capsule medical apparatus according to claim 17, wherein a time bar is displayed by assuming that a start point is time for observing the first portion and an end point is time for observing the second portion, and time for obtaining the displayed vital information is displayed onto the time bar.

19. A capsule medical apparatus according to claim 1 comprising:
a magnet arranged to the capsule;
a position detecting unit which detects a position of the capsule;
a magnetic field generating unit which generates rotating magnetic field;
a magnetic field changing unit which changes the direction of the rotating magnetic field;
an operating-direction converting unit arranged to the capsule; and
a control unit which detects the amount of movement based on an output from the position detecting unit and operates the magnetic field changing unit.

20. A capsule medical apparatus according to claim 19, wherein when the amount of movement of the capsule is smaller than a reference amount, the control unit operates the magnetic field changing unit and generates the rotating magnetic field from the magnetic field generating unit.

21. A capsule medical apparatus according to claim 1 comprising:
a magnet arranged to the capsule;
a position detecting unit which detects a position of the capsule;
a magnetic field generating unit which generates rotating magnetic field;
a magnetic field changing unit which changes the direction of the rotating magnetic field;
an operating-direction converting unit structured with winding spiral projections arranged on an outer surface of the capsule;
a space setting unit; and
a control unit which estimates a relationship between the space set by the space setting unit and the capsule position detected by the position detecting unit and operates the magnetic field changing unit.

22. A capsule medical apparatus according to claim 1 comprising:
a magnet arranged to the capsule;
a position detecting unit which detects a position of the capsule;
a magnetic field generating unit which generates rotating magnetic field;
a magnetic field changing unit which changes the direction of the rotating magnetic field;
an operating-direction converting unit arranged to the capsule; and
a control unit which controls a timing for operating the position detecting unit and a timing for operating the magnetic field changing unit.

23. A capsule medical apparatus comprising a capsule having an image pick-up unit for capturing a vital image, inserted or swallowed in vivo, and an extracorporeal unit arranged in vitro, the capsule medical apparatus comprising:
a magnet arranged to the capsule;
a magnetic field generating unit which generates rotating magnetic field;
a magnetic field changing unit which changes the direction of the rotating magnetic field;
an operating-direction converting unit arranged to the capsule;
an image processing unit which detects the amount of movement based on a plurality of outputs from the image pick-up unit; and
a control unit which operates the magnetic field changing unit based on the image processing unit.

24. A capsule medical apparatus comprising a capsule having a vital information detecting unit for obtaining vital information, inserted or swallowed in vivo, and an extracorporeal unit arranged in vitro, the capsule medical apparatus comprising:
a setting unit which sets a portion of the internal organ previously before the capsule is inserted or swallowed in vivo; and
a display unit which displays by a ratio of time division, observation information between a first portion set by the setting unit and a second portion set by the setting unit by a ratio of time division.

25. A capsule medical apparatus according to claim 24, wherein the setting unit comprises:
a position detecting unit which detects a position of the capsule;
a spatial setting unit which designates a space indicating the first portion and the second portion from the space for detecting the position of the capsule by the position detecting unit; and
a comparing unit which compares capsule position information from the position detecting unit with the space set by the space setting unit and outputs a signal, and
wherein the comparing unit comprises a storing unit which records time when the comparing unit issues the signal.

26. A capsule medical apparatus according to claim 24, wherein the setting unit comprises an image processing unit which processes the image obtained by the capsule and detects the first portion and the second portion.

27. A capsule medical apparatus according to claim 24, wherein the image processing unit comprises an input unit which inputs the first portion and the second portion while the observation information from the capsule is displayed.

28. A capsule medical apparatus according to claim 25, wherein the image processing unit comprises an input unit which inputs the first portion and the second portion while the observation information from the capsule is displayed.

29. A capsule medical apparatus according to claim 26, wherein the image processing unit comprises an input unit which inputs the first portion and the second portion while the observation information from the capsule is displayed.

30. A capsule medical apparatus according to claim 26, wherein the image processing unit performs determination by extracting the amount of characteristics.

31. A capsule medical apparatus according to claim 26, wherein the image processing unit performs determination by calculating a correlation with an image database.

32. A control method for a capsule medical apparatus having a vital information detecting unit for obtaining vital information, inserted or swallowed in vivo, the method comprising the steps of:
designating a space in vivo previously before the capsule is inserted or swallowed in vivo;
detecting a position in vivo of a capsule medical apparatus;
determining whether or not the detected position is overlapped to the space; and
deciding a state of the capsule medical apparatus based on the determination.
